(19) United States
(12) Patent Application Publication
(10) Pub. No.: US 2014/0165228 A1
(43) Pub. Date: Jun. 12, 2014

(54) IDENTIFICATION OF DIURNAL RHYTHMS IN PHOTOSYNTHETIC AND NON-PHOTOSYNTHETIC TISSUES FROM ZEA MAYS AND USE IN IMPROVING CROP PLANTS

(71) Applicants: E.I. DUPONT de NEMOURS, Wilmington, DE (US); PIONEER HI BRED INTERNATIONAL INC, Johnston, IA (US)

(72) Inventors: OLGA DANILEVSKAYA, Johnston, IA (US); Jeffrey E. Habben, Urbandale, IA (US); Kevin Hayes, Urbandale, IA (US); Carl Simmons, Des Moines, IA (US); Stephane Deschamps, Hockessin, DE (US)

(73) Assignees: E.I. DUPONT de NEMOURS, Wilmington, DE (US); PIONEER HI BRED INTERNATIONAL INC, Johnston, IA (US)

(21) Appl. No.: 14/087,036

(22) Filed: Nov. 22, 2013

Related U.S. Application Data
(63) Continuation of application No. 12/985,413, filed on Jan. 6, 2011.
(60) Provisional application No. 61/292,572, filed on Jan. 6, 2010.
(60) Provisional application No. 61/302,389, filed on Feb. 8, 2010.
(60) Provisional application No. 61/362,382, filed on Jul. 8, 2010.

(51) Int. Cl. C12Q 1/68; A01H 5/00; A01H 5/10; C12N 15/82

(52) U.S. Cl. CPC C12Q 1/6802; C12N 15/82; A01H 5/00; A01H 5/10; C12N 15/8273; C12N 15/8286; C12N 15/8279
USPC 800/289; 435/419; 435/320.1; 506/9; 530/300; 536/23.1; 800/278; 800/290; 800/298; 800/312; 800/314; 800/317.4; 800/320; 800/320.1; 800/320.2; 800/320.3; 800/322

(57) ABSTRACT

The present disclosure provides polynucleotide sequences relating to the diurnal cycling in maize leaf and ear tissues. The disclosure provides polynucleotide sequences and the use of encoded polypeptides associated with the oscillation. The disclosed sequences are responsible for controlling plant growth, source-sink relationships and yield in crop plants.

### CROSS REFERENCE

This utility application is a continuation of U.S. patent application Ser. No. 12/985,413 filed Jan. 6, 2011 and claims the benefit U.S. Provisional Application No. 61/292,572, filed Jan. 6, 2010, U.S. Provisional Application No. 61/302,389, filed Feb. 8, 2010 and U.S. Provisional Application No. 61/362,382, filed Jul. 8, 2010, all of which are incorporated herein by reference.

### FIELD OF THE INVENTION

The disclosure relates generally to the field of molecular biology.

### BACKGROUND OF THE INVENTION

The day-night cycle is a major environmental cue that controls daily and seasonal rhythms in plants. Diurnal light-dark transitions entrain the internal circadian clock that generates rhythms that are self-sustained (free-running) under constant light conditions. A simplified model of the clock is comprised by three basic components: an input pathway that senses light; a core oscillator that is the transcriptional machinery generating rhythms; and output pathways that control various developmental and metabolic processes, resulting in the appropriate physiological adaptations to the day-night cycle (Barak, et al., (2000) Trends Plant Sci 5:517-522; Harmer, (2009) Annu Rev Plant Biol 60:357-377). The proper synchronization of the internal clock and external light/dark cycles result in better plant fitness, survival, competitive advantage (Dodd, et al., (2005) Science 309:630-633) and growth vigor (Ni, et al., (2009) Nature 457:327-331).

The genetic architecture of the plant circadian system has thus far been mostly elucidated in Arabidopsis (Mas, (2008) Trends Cell Biol 18:273-281). The input pathways are comprised of two sets of photoreceptors, the red/far-red sensing phytochromes (PHYA-E) and the UV-A/blue-light sensing cryptochromes (CRY1 and CRY2), which percept light during the day and send signals to the core oscillator (Nemhauser, (2008) Curr Opin Plant Biol 11:4-8). The core oscillator genes form interlocking transcriptional feedback loops (Harmer and McClung, (2009) Science 323:1440-1441). The morning loop, consists of the MYB-like transcription factors CCA1 (CIRCADIAN CLOCK ASSOCIATED) and LHY (LATE ELONGATED HYPOCOTYL), which participate in regulation of two different loops. In the morning loop, CCA1/LHY negatively regulate transcription of the pseudo-response regulator TOC1 (TIMING OF CAB EXPRESSION 1) and the TOP-like transcription factor CHE (CCA1 HIKING EXPEDITION). TOC1/CHE form a complex that positively regulates transcription of CCA1/LHY (Pruneda-Paz, et al., (2009) Science 323:1481-1485). In the day loop, CCA1/LHY positively regulates transcription of the PRR7 and PRR9 (PSEUDO-RESPONSE REGULATORS) both of which negatively regulate CCA1/LHY. In the evening loop, TOC1/CHE works as a negative regulator of GI (GIGANTIA), itself a positive regulator of TOC1. The evening gene ZTL (ZEITLUPE, a protein-degrading F-box protein), involved in degradation of TOC1 and PRR3 proteins, provides regulation of the core clock components at the protein level (Mas, et al., (2003) Nature 426:567-570). The multiple interlocking transcription loops maintain a robust yet flexible genetic machinery (Harmer (2009) supra)

The circadian clock generates rhythmic outputs that regulate many plant developmental and physiological processes including: growth (Nozue, et al., (2007) Nature 448:358-361; Nozue and Maloof, (2006) Plant Cell Environ 29:396-408), flowering time, tuberization in annuals, growth cessation and bud set in perennials (Lagercrantz, (2009) J Exp Bot 60:2501-2515), photosynthesis (Sun, et al., (2003) Plant Mol Biol 53:467-478), nitrogen uptake (Gutierrez, et al., (2008) Proc Natl Acad Sci USA 105:4939-4944) and hormone signaling and stress response (Covington and Harmer, (2007) PLoS Biol 5:e222). However, knowledge of the molecular nodes that link the circadian clock with output pathways are just now emerging. So far the best understood connection is the photoperiod regulation of flowering time in Arabidopsis and rice. The Arabidopsis clock gene GI and its rice homologue OsGI promotes expression of the transcription factors CO (CONSTANS) and OsCO (Hd1, HEADING1), which control transcription of the downstream floral activator FT (FLOWERING LOCUS T) in Arabidopsis and its homologous gene Hd3a (HEADING 3a) in rice (Michaels, (2009) Curr Opin Plant Biol 12:75-80, Tsuji and Komiya, (2008) Rice 1:25-35). The photoperiod sensitive pathways ensure flowering under favorable conditions.

Several publications identified molecular connections between the Arabidopsis core oscillators and a broad range of plant physiological processes. Rhythmic hypocotyl growth is promoted by positive action of two basic helix-loop-helix transcription factors, PIF4 and PIF5 (PHYTOCHROM-INERACTING FACTOR) whose transcript levels are regulated by CCA1 (Nozue, et al., (2007) Nature 448:358-361). The hypocotyl growth is also independently regulated by free levels of the phytohormone auxin, produced by the auxin biosynthetic gene YUCCAS, that is controlled directly by the clock-dependent Myb-like transcription factor RVE1 (REVEILLE 1) (Rawat, et al., (2009) Proc Natl Acad Sci USA 106:16883-16888). This is a direct link between circadian oscillators and the auxin networks that coordinate seedling growth in Arabidopsis. Output pathways of PPR9/7/5 genes are related to maintenance of the central metabolism, mainly in mitochondria, and in particular the tricarboxylic acid (TCA) cycle (Fukushima, et al., (2009) Proc Natl Acad Sci USA 106:7251-7256). TOC1 is also linked with the stress-related ABA hormone connecting the circadian clocks with plant responses to drought (Legnaioli, et al., (2009) The EMBO Journal 28:3745-3757).

The use of microarray technology has uncovered the pervasive influence of circadian rhythms on gene transcription in Arabidopsis. These studies have mainly focused on light-sensing tissues, such as Arabidopsis rosettes. Up to 35% of Arabidopsis genes are circadian-regulated in green tissues (Covington, et al., (2008) Genome Biol 9:R130; Harmer, et al., (2000) Science 290:2110-2113; Ptitsyn, (2008) BMC Bioinformatics 9(9):S18). While animal models have shown that nearly every tissue has a large circadian component to its transcriptional program, diverse plant tissues have not yet been systematically evaluated as to the relative contribution of diurnal light cycles on transcription (Ptitsyn, et al., (2006) PLoS Comput Biol 2:e16). In the pre-genomic era diurnal changes were observed in maize leaf photosynthesis and leaf elongation rates, which were the greatest at midday (Kalt-Torres and Huber, (1987) Plant Physiol 83:294-298, Kalt-Torres, et al., (1987) Plant Physiol 83:283-288, Usuda, et al., (1987) Plant Physiol 83:289-293). Diurnal oscillation of the endosperm-specific transcription factor O2 (Opaque 2) was also found in non-photosynthetic kernels, and it was proposed that O2 activity is controlled by diurnal metabolite flux (Ciceri, et al., (1999) Plant Physiol 121:1321-1328). Diurnal and circadian rhythms were demonstrated for maize homologues of GI (gigz1) and CO (conz1), which are direct outputs of the circadian clock in the photoperiod pathway controlling Arabidopsis flowering time (Miller, et al., (2008) Planta 227:1377-1388), even though temperate maize is a day-neutral plant whose flowering is not regulated by the day length.

This study identified two TOC1 homologues, ZmTOCa and ZmTOCb, which mapped to chromosome 5 and 4, respectively. Transcription of both genes peaks at 6 pm, consistent with Arabidopsis TOC1 gene expression. TOC1 is a member of the pseudo-response regulator (PRR) family composed of evolutionarily conserved five PRR genes in Arabidopsis and rice (Murakami, et al., (2007) Biosci Biotechnol Biochem 71:1107-1110; Murakami, et al., (2003) Plant Cell Physiol 44:1229-1236). In addition to two ZmTOC1 homologues, the study also identified ZmPRR73, ZmPRR37 and ZmPRR59 that were named after rice PRR genes based on the level of sequence similarly (Murakami, et al., (2003)). Also identified were two ZEITLUPE homologues (Kim, et al., (2007) Nature 449:356-360), ZmZTLa and ZmZTLb, which mapped to chromosome 2 and 4. Two maize orthologs of GIGANTIA, gigz1A and gigz1B, were described previously (Miller, et al., (2008) Planta 227:1377-1388) and are here confirmed their oscillation in both ears and leaves. The majority of the known core components cycle in both Agilent (Agilent Technologies, Inc., Life Sciences and Chemical Analysis, 2850 Centerville Road, Wilmington, Del. 19808-1610, USA) and Illumina (Illumina, Inc., 9885 Towne Centre Drive, San Diego, Calif. 92121 USA), analyses. Cycling of the core components ZmCCA, ZmLHY, ZmTOC1a and ZmTOC1b were further confirmed via RT-PCR analysis. The amplitudes of the core components is attenuated in the developing ear when compared with leaf tissue, but still robust. These data show that the majority of the plant core oscillator system is functioning in non-photosynthetic tissues such as ear, but the oscillator output is apparently largely isolated from the transcriptional machinery affecting downstream diurnal expression changes.

Components of the core clock mechanism and proximal signaling mechanism emanating from it, could be modified in such manner as to positively affect crop performance, as by for example shifting or extending the relationship between sources and sinks such as leaves and ears. Wholesale genetic complementation of diurnal patterns from different germplasm sources has been shown to augment the combined diurnal patterns and apparent fitness (Ni, (2009) supra).

### BRIEF DESCRIPTION OF THE DRAWINGS

FIG. 1: Diurnal Core Clock Components functioning in maize, chromosome location and time of peak expression levels.

FIG. 2 (as FIG. 2A and FIG. 2B): Validation of diurnal expression for ZmCCA1, ZmLHY, ZmTOC1a and ZmTOC1b by qRT-PCR.

FIG. 3: Diurnal expressed genes in ears, chromosome location and time of peak expression levels.

FIG. 4: Exon/Intron structures of ZmCCA1 and ZmLHY genes

FIG. 5: Diurnal Patterns for Temporally Enriched Gene Functional Terms

### BRIEF SUMMARY OF THE INVENTION

No systematic study of diurnal/circadian transcriptional patterns in maize has yet been undertaken. The present study was initiated to examine the extent that the diurnal cycle plays in regulating gene transcription in maize using modern genome-wide profiling technologies. Field experiments were designed under natural undisturbed conditions and sampled both a photosynthetic tissue, leaf and a non-photosynthetic tissue, developing ear. Thousands of transcripts that markedly cycle in the maize leaves were identified. In non-photosynthetic ears however just a small set of genes, as little as 45, were clearly diurnally cycling. Many of these are maize homologues of Arabidopsis core oscillator genes, indicating that core circadian genes are conserved in maize and diurnally expressed in both photosynthetic and non-photosynthetic tissues.

A number of maize diurnally regulated genes were identified during the analyses. A total of 471 sequences, including those from immature ear, those having high amplitude/magnitude cycling in leaf tissue and diverse sequences associated with NUE and Carbon::Nitrogen functions. The sequences contain ORFs, encoded polypeptides, and their associated promoters.

The following list includes some of the embodiments of the disclosure:
1. An isolated polynucleotide selected from the group consisting of:
   a. a polynucleotide having at least 90% sequence identity, as determined by the GAP algorithm under default parameters, to the full length sequence of a polynucleotide selected from the group consisting of SEQ ID NOS: 1, 2, 3, 4, 5, 6, 7, 8, 20, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470; wherein the polynucleotide encodes a polypeptide that functions as a modifier of diurnal activity;
   b. a polynucleotide selected from the group consisting of SEQ ID NOS: 1, 2, 3, 4, 5, 6, 7, 8, 20, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470;
   c. a polynucleotide which is fully complementary to the polynucleotide of (a) or (b);
   d. a polypeptide encoded by the polynucleotide of (a) or (b); and
   e. a polypeptide having at least 90% sequence identity, as determined by the GAP algorithm under default parameters, to the full length sequence of a polypeptide selected from the group consisting of SEQ ID NOS; 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 467, 469 and 471.
2. A recombinant expression cassette, comprising the polynucleotide of claim 1, wherein the polynucleotide is operably linked, in sense or anti-sense orientation, to a promoter.
3. A host cell comprising the expression cassette of claim 2.
4. A transgenic plant comprising the recombinant expression cassette of claim 2.
5. The transgenic plant of claim 4, wherein said plant is a monocot.
6. The transgenic plant of claim 4, wherein said plant is a dicot.
7. The transgenic plant of claim 4, wherein said plant is selected from the group consisting of: maize, soybean, sunflower, sorghum, canola, wheat, alfalfa, cotton, rice, barley, millet, peanut, sugar cane and cocoa.
8. A transgenic seed from the transgenic plant of claim 4.
9. A method of modulating diurnal rhythm in plants, comprising:
   a. introducing into a plant cell a recombinant expression cassette comprising the polynucleotide of claim 1 operably linked to a promoter; and
   b. culturing the plant under plant cell growing conditions; wherein the diurnal in said plant cell is modulated.
10. The method of claim 9, wherein the plant cell is from a plant selected from the group consisting of: maize, soybean, sunflower, sorghum, canola, wheat, alfalfa, cotton, rice, barley, millet, peanut, sugar cane and cocoa.
11. A method of modulating the whole plant or diurnal rhythm in a plant, comprising:
   a. introducing into a plant cell a recombinant expression cassette comprising the polynucleotide of claim 1 operably linked to a promoter;
   b. culturing the plant cell under plant cell growing conditions; and
   c. regenerating a plant form said plant cell; wherein the diurnal rhythm in said plant is modulated.
12. The method of claim 11, wherein the plant is selected from the group consisting of: maize, soybean, sorghum, canola, wheat, alfalfa, cotton, rice, barley, millet, peanut and cocoa.
13. A product derived from the method of processing of transgenic plant tissues expressing an isolated polynucleotide encoding a diurnally functioning gene, the method comprising:
   a. transforming a plant cell with a recombinant expression cassette comprising a polynucleotide having at least 90% sequence identity to the full length sequence of a polynucleotide selected from the group consisting of SEQ ID NO: 1, 2, 3, 4, 5, 6, 7, 8, 20, 40, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470; operably linked to a promoter; and
   b. culturing the transformed plant cell under plant cell growing conditions; wherein the growth in said transformed plant cell is modulated;
   c. growing the plant cell under plant-forming conditions to express the polynucleotide in the plant tissue; and
   d. processing the plant tissue to obtain a product.
14. The transgenic plant of claim 13, wherein the plant is a monocot.
15. The transgenic plant of claim 13, wherein the plant is selected from the group consisting of: maize, soybean, sunflower, sorghum, canola, wheat, alfalfa, cotton, rice, barley, sugar cane and millet.
16. The transgenic plant of claim 4, where overexpression of the polynucleotide leads to which has improved plant growth as compared to non-transformed plants.
17. The transgenic plant of claim 4, where the plant exhibits improved source-sink relationships as compared to non-transformed plants.
18. The transgenic plant of claim 4, where the plant has improved yield as compared to non-transformed plants.
19. A regulatory polynucleotide molecule comprising a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.
20. A chimeric polynucleotide molecule comprising the nucleic acid fragment of claim 19.
21. The chimeric molecule of claim 20 comprises the diurnal regulatory element and a tissue specific expression element.
22. The chimeric molecule of claim 21, wherein the tissue specific expression element is selected from the group consisting of root specific, bundle sheath cell specific, leaf specific and embryo specific.
23. The regulatory polynucleotide molecule of claim 19, wherein said regulatory polynucleotide molecule is a promoter.
24. A construct comprising the regulatory molecule of claim 19 operably linked to a heterologous polynucleotide molecule, wherein the heterologous molecule confers a trait of interest.
25. The construct of claim 24, wherein the trait of interest is selected from the group consisting of drought tolerance, freezing tolerance, chilling or cold tolerance, disease resistance and insect resistance.
26. The construct of claim 24, wherein the heterologous molecule functions in source-sink metabolism.
27. A transgenic plant transformed with the regulatory molecule of claim 19.
28. The transgenic plant of claim 27 is monocotyledonous.
29. The transgenic plant of claim 27 is selected from the group consisting of maize, soybean, canola, cotton, sunflower, alfalfa, sugar beet, wheat, rye, rice, sugarcane, oat, barley, turf grass, sorghum, millet, tomato, pigeon pea, vegetable, fruit tree and forage grass.
30. A method of increasing yield of a plant, the method comprising expressing a heterologous polynucleotide of interest under the control of the regulatory molecule of claim 19.
31. The method of claim 30, wherein the heterologous polynucleotide is a diurnally regulated plant gene.
32. A method of increasing abiotic stress tolerance in a plant, the method comprising expressing one or more polynucleotides that confer abiotic stress tolerance in plants under the control of the regulatory molecule of claim 19.
33. The method of claim 32, wherein the abiotic stress tolerance is selected from the group consisting of drought tolerance, freezing tolerance and chilling or cold tolerance.
34. The method of claim 33, wherein the polynucleotide that confers drought tolerance is expressed under the control of a regulatory element whose peak expression is around mid-day or late afternoon.
35. The method of claim 33, wherein the polynucleotide that confers freezing or cold tolerance is expressed under the control of a regulatory element whose peak expression is around dawn or mid-morning.
36. A method of reducing yield drag of transgenic gene expression, the method comprising expressing a transgene operably linked to a regulatory polynucleotide molecule comprising a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.
37. A method of screening for gene candidates involved in abiotic stress tolerance, the method comprising (a) identifying one or more gene candidates that exhibit yield drag under constitutive or tissue specific expression and (b) expressing the gene candidates under the control of the a regulatory molecule that directs diurnal expression pattern.
38. The method of claim 37, wherein the regulatory molecule comprises a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.

### DETAILED DESCRIPTION OF THE INVENTION

Unless defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the disclosure.

The present disclosures now will be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present disclosure will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984) and the series METHODS IN ENZYMOLOGY, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present disclosure, the following terms will be employed and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., DIAGNOSTIC MOLECULAR MICROBIOLOGY: PRINCIPLES AND APPLICATIONS, Persing, et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present disclosure, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present disclosure may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray, et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “diurnal nucleic acid” means a nucleic acid comprising a polynucleotide (“diurnal polynucleotide”) encoding a diurnal polypeptide.

As used herein, “nucleic acid” includes reference to a deoxyribonucleotide or ribonucleotide polymer in either single- or double-stranded form and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989) and CURRENT PROTOCOLS IN MOLECULAR BIOLOGY, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds suspension cultures, embryos, meristematic regions, callus tissue, leaves, roots, shoots, gametophytes, sporophytes, pollen and microspores. The class of plants, which can be used in the methods of the disclosure, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” includes reference to bushels per acre of a grain crop at harvest, as adjusted for grain moisture (15% typically). Grain moisture is measured in the grain at harvest. The adjusted test weight of grain is determined to be the weight in pounds per bushel, adjusted for grain moisture level at harvest. As used herein, improved “source-sink” relationship includes reference to a trait associated with an improvement of the ratio of assimilate supply (i.e., source) and demand (i.e., sink) during grain filling.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins (e.g., transcription factors) to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions.

As used herein, “regulatory element” or “regulatory polynucleotide” refers to nucleic acid fragment that modulates the expression of a transcribable polynucleotide that is associated with the regulatory element. Such association can occur in cis. A plant promoter can also be used as a regulatory element for modulating the expression of a particular gene or genes that are operably associated to the promoters. When operably associated to a transcribable polynucleotide molecule, a regulatory element affects the transcriptional pattern of the transcribable polynucleotide molecule. “cis-element” or “cis-acting element” refers to a cis-acting transcriptional regulatory element that affects gene expression. A cis-element may function to bind transcription factors, trans-acting proteins that modulate transcription. The diurnal promoters disclosed herein may contain one or more cis-elements that provide diurnal gene expression pattern.

The plant promoters and the regulatory elements disclosed herein can include nucleotide sequences generated by promoter engineering, i.e., combination of known promoters and/or regulatory elements to produce artificial, synthetic, chimeric or hybrid promoters. Such promoters can also combine cis-elements from one or more promoters, for example, by adding a heterologous tissue specific regulatory element to a promoter that contains diurnal expression regulatory elements. Thus, the design, construction, and use of chimeric or hybrid promoters comprising at least one cis-element of the promoters disclosed herein for modulating the expression of operably linked polynucleotide sequences is contemplated.

The promoter sequences disclosed herein including SEQ ID NOS: 31-183 and fragments there of that include for example, 50, 100, 150, 200, 300, 400, 500, 600, 700, 800, 900, 1000, 1100, 1200, 1300, 1400, 1500, 1600, 1700, 1800, 1900, 2000 and up to 2500 contiguous nucleotides thereof and about 80% or 85% or 90% or 95% or 99% identical to those fragments are contemplated for use in modulating the expression pattern of one or more heterologous genes. The term “heterologous” in this context means that the expression of the nucleotide of interest is modulated by a promoter sequence or a fragment thereof that is not the nucleotide's own promoter. Deletion constructs of the various promoter sequences disclosed herein are readily made by one of ordinary skill in the art following the guidance provided herein. About 25-50 contiguous nucleotides that flank the 3′ or the 5′ ends of the disclosed regulatory elements are selected for modulation of gene expression. Mutational analysis are also performed to enhance the specificity of diurnal regulation.

The term “diurnal polypeptide” refers to one or more amino acid sequences. The term is also inclusive of fragments, variants, homologs, alleles or precursors (e.g., preproproteins or proproteins) thereof. A “diurnal protein” comprises a diurnal polypeptide. Unless otherwise stated, the term “diurnal nucleic acid” means a nucleic acid comprising a polynucleotide (“diurnal polynucleotide”) encoding a diurnal polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus or nucleic acid fragment. Typically, the recombinant expression cassette portion of an expression vector includes, among other sequences, a nucleic acid to be transcribed and a promoter.

The terms “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C. and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-84: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” Elsevier, New York (1993) and CURRENT PROTOCOLS IN MOLECULAR BIOLOGY, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinylpyrrolidone, 5 g bovine serum albumin in 500 ml of water), 0.1 mg/ml boiled salmon sperm DNA and 25 mM Na phosphate at 65° C. and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may conduct optimal alignment of sequences for comparison; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package®, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65, and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package® are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package® is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The disclosure discloses diurnal polynucleotides and polypeptides. The novel nucleotides and proteins of the disclosure have an expression pattern which indicates that they regulate cell number and thus play an important role in plant development. The polynucleotides are expressed in various plant tissues. The polynucleotides and polypeptides thus provide an opportunity to manipulate plant development to alter seed and vegetative tissue development, timing or composition. This may be used to create a sterile plant, a seedless plant or a plant with altered endosperm composition.

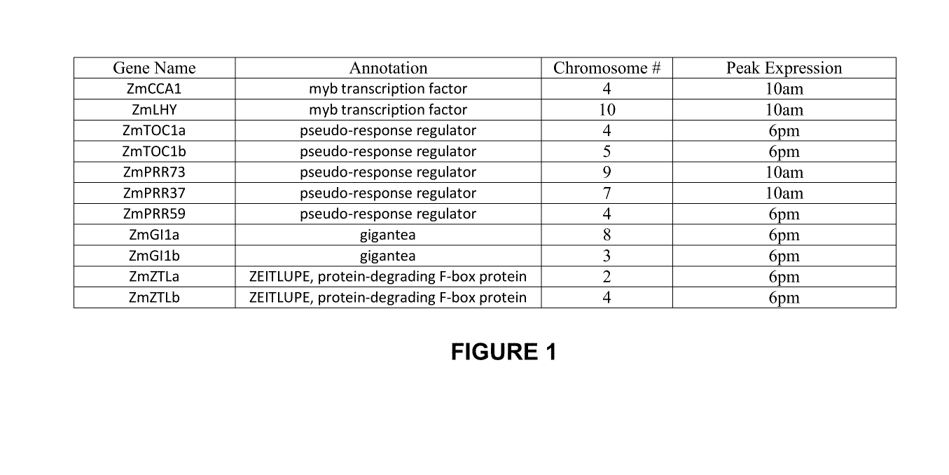

Maize orthologs of the Arabidopsis and rice circadian genes were identified by reciprocal BLAST searches plus evaluation of whether the inferred proteins relationships abide by the speciation pattern, and then queried for oscillation patterns in leaf and ear tissues. Employing these criteria, maize homologues were identified for several major core components including CCA1/LHY, TOC1, PRR7/3, GI and ZTL (FIG. 1).

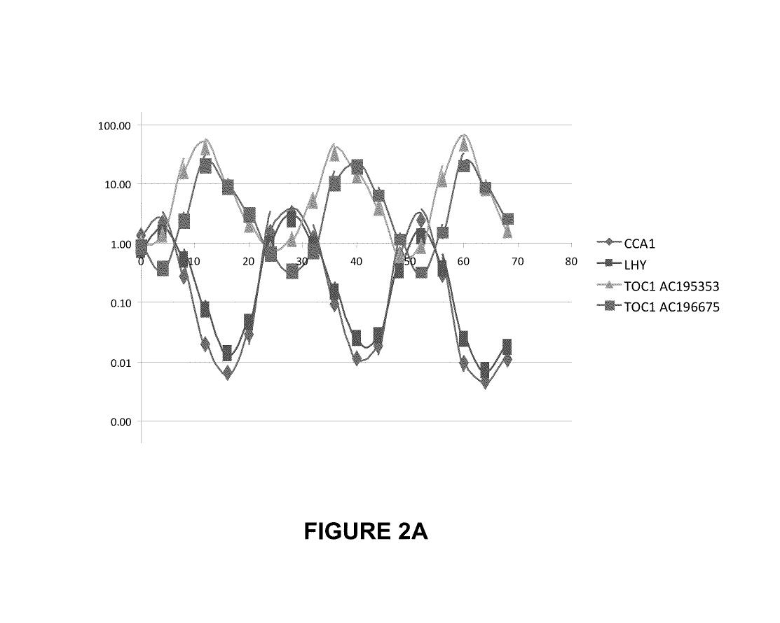

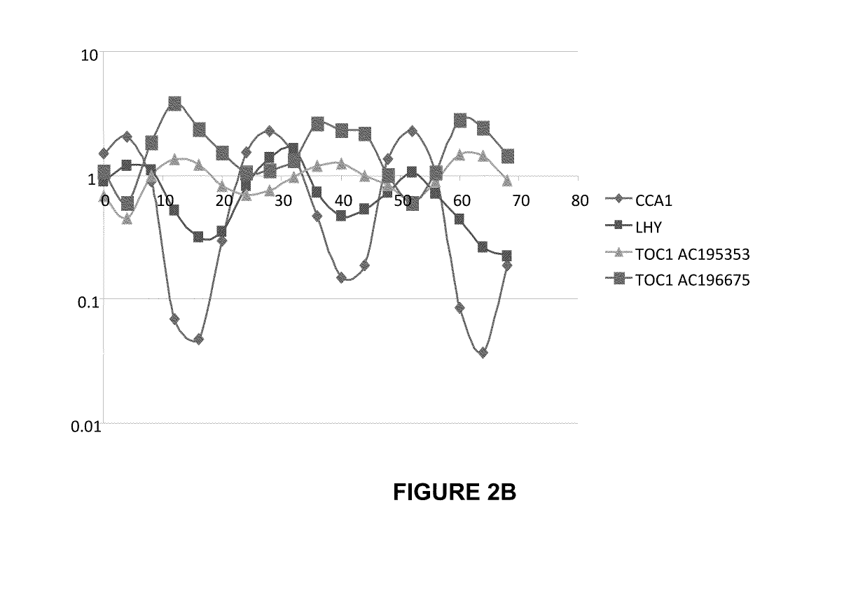

This study identified two TOC1 homologues, ZmTOCa and ZmTOCb, which mapped to chromosome 5 and 4, respectively. Transcription of both genes peaks at 6 pm, consistent with Arabidopsis TOC1 gene expression. TOC1 is a member of the pseudo-response regulator (PRR) family composed of evolutionarily conserved five PRR genes in Arabidopsis and rice (Murakami, et al., (2007) Biosci Biotechnol Biochem 71:1107-1110; Murakami, et al., (2003) Plant Cell Physiol 44:1229-1236). In addition to two ZmTOC1 homologues, the study also identified ZmPRR73, ZmPRR37 and ZmPRR59 that were named after rice PRR genes based on the level of sequence similarly (Murakami, et al., (2003)). Also identified were two ZEITLUPE homologues (Kim, et al., (2007) Nature 449:356-360), ZmZTLa and ZmZTLb, which mapped to chromosome 2 and 4. Two maize orthologs of GIGANTIA, gigz1A and gigz1B, were described previously (Miller, et al., (2008) Planta 227:1377-1388) and are here confirmed their oscillation in both ears and leaves. Cycling of the core components ZmCCA, ZmLHY, ZmTOC1a and ZmTOC1b were further confirmed via RT-PCR analysis (FIG. 2). The amplitudes of the core components is attenuated in the developing ear when compared with leaf tissue, but still robust. These data show that the majority of the plant core oscillator system is functioning in non-photosynthetic tissues such as ear, but the oscillator output is apparently largely isolated from the transcriptional machinery affecting downstream diurnal expression changes.

It was determined that diurnally regulated transcripts pervade most functions of the maize leaf cells. The 6674 transcripts (out of 10,037 Agilent array probes) that are here determined to be diurnally regulated represent over 22% of the total detected transcripts expressed and these 6674 transcripts could be assigned to 1716 different Gene Ontology (GO) terms and 22 KOGs functional categories.

Generally, individual genes peak have just one peak in their diurnal cycle. When these genes were assigned to functional terms and the relative enrichment of those functional terms was plotted across the span of the day, most functions had a marked enrichment for a time particular pattern in the day. There was also however a clear tendency for some functional terms to have a bimodal pattern, wherein there was a mid-morning peak at 10 AM and a secondary peak in the late afternoon or evening at 6 PM or 10 PM. Over 18% of the functional terms were classified as bimodal regulated, with further subdivisions made according to relative enrichment of the morning or afternoon peak. Together with the functions assigned as peaking at just one peak in the day, 94.5% of the 1738 functions were assigned to one of these patterns, with just 95 leftover to be assigned to the “Other” patterns.

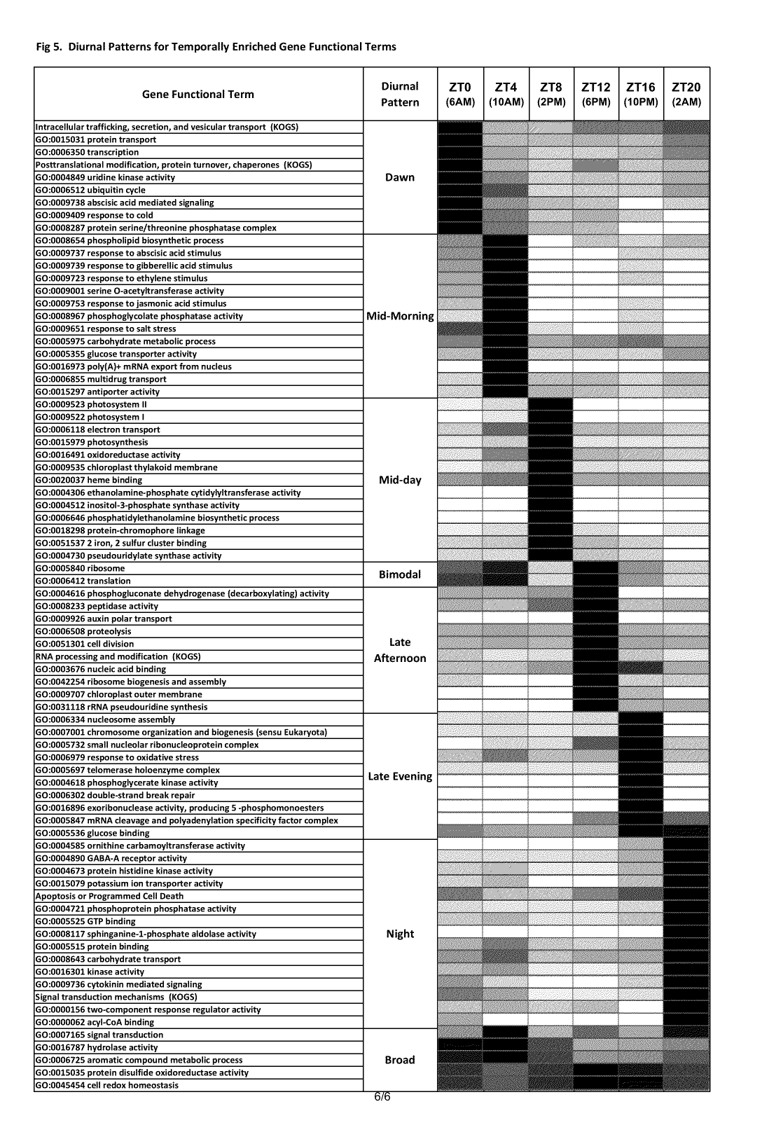

Often the bimodal patterned functional terms represent broader gene-rich functional classifications such as protein kinase activity, signal transduction mechanism, or amino acid transport and metabolism. (FIG. 5) Accordingly, these bimodal patterns tend to also have fair representation across the day and not just at 10 AM and 6/10 PM. Nonetheless, it remains a chief feature of the diurnal pattern that gene and functional enrichment peaks typically occur in the mid-morning and again later in the afternoon/evening. In this experiment the sunrise was 6:02 AM and the sunset at 8:40 PM. The sunrise is thus 4 hours before the 10 AM functional peak, but the sunset is 2.45 hours after the 6 PM timepoint and 1.25 hours before the 10 PM timepoint. Additional timepoints may provide greater resolution, but that among the bimodal patterns the 10 AM>6/10 PM patterns have over 70% higher functional enrichment indexes than the 6 PM>10 AM patterns may relate to this asymmetrical placement of the timepoints relative to the sunrise and sunset. Alternatively, some functional classes may be inherently enriched for the morning phase, reflecting underlying biological tendencies.

That 1643 or 94.5% of the functional terms were assigned to one temporal peak pattern indicates a fairly defined progression of functions across the day. Functional groups are thus not uniformly spread across the different phases of the day, but instead exhibit distinct patterns and biases. The dawn enriched functional categories include for example: response to cold, lipid catabolism and hormone signaling. This follows by mid-morning with multiple hormone response functions becoming enriched. The mid-day becomes dominated expectedly by photosynthesis systems I and II, chlorophyll synthesis, and monodehydroascorbate reductase (MDAR) involved in antioxidant generation. Late afternoon and evening reveal a marked enrichment for ribosomal and DNA damage repair, including helicase, telomerase and endonuclease activity, suggesting chromosomal and ribosomal repair systems are activated. In addition sucrose transport and the pentose-phosphate shunt peak in late afternoon/evening suggesting dynamics of chloroplast carbohydrate metabolism. Late evening peaks include the red::far-red light phototransduction, noted in the introduction as regulating the core clock, but also hydrogen peroxide metabolism. At night caspase(-like) activity, often associated with cell death, photosystem II catabolism, nucleotide transport and metabolism and acyl-CoA binding functions all peak. Other irregular but interesting peak patterns are amino acid glycosylation cresting at both 6 PM and 2 AM, and both malic enzyme and calmodulin binding peaking at 10 AM and 2 AM. These are just a few examples of a very complex story addressing the whole plant cellular physiology.

Notably, despite the great variety of genes and functions being diurnally regulated, most functional categories have only a minority of members that are diurnally regulated. Among the 1738 functional categories, the mean coverage was 28.2% with the median 20% and mode about 15%. Functional categories containing multiple genes were not completely represented by diurnally regulated transcripts, and few functional categories were outstandingly enriched for diurnally regulated transcripts. GO:0004614 phosphoglucomutase activity had five of six and GO:0009926 auxin polar transport had three of four, transcripts among the diurnal set. These findings indicate that diurnally regulated transcripts are within but do not dominate these diverse functions.

A number of maize diurnally regulated genes were identified during the analyses. A total of 471 sequences, including those from immature ear, those having high amplitude/magnitude cycling in leaf tissue, and diverse sequences associated with NUE and Carbon::Nitrogen functions. The sequences contain ORFs, encoded polypeptides and their associated promoters.

### Nucleic Acids

The present disclosure provides, inter alia, isolated nucleic acids of RNA, DNA and analogs and/or chimeras thereof, comprising a diurnal polynucleotide.

The present disclosure also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 4 of Murray, et al., supra.

The diurnal nucleic acids of the present disclosure comprise isolated diurnal polynucleotides which are inclusive of:
(a) a polynucleotide encoding a diurnal polypeptide and conservatively modified and polymorphic variants thereof;
(b) a polynucleotide having at least 70% sequence identity with polynucleotides of (a) or (b);
(c) complementary sequences of polynucleotides of (a) or (b).
   The following table, Table 1, lists the specific identities of the polynucleotides and polypeptides and disclosed herein.

TABLE 1

| | | Polynucleotide/ | |
|---|---|---|---|
| Name | Plant species | Polypeptide | SEQ ID NO: |
| ZmTOC1b | Zea mays | Polynucleotide | SEQ ID NO: 1 |
| ZmMYB.L | Zea mays | Polynucleotide | SEQ ID NO: 2 |
| ZmZTLa | Zea mays | Polynucleotide | SEQ ID NO: 3 |
| ZmZTLb | Zea mays | Polynucleotide | SEQ ID NO: 4 |
| ZmPRR37 | Zea mays | Polynucleotide | SEQ ID NO: 5 |
| ZmPRR59 | Zea mays | Polynucleotide | SEQ ID NO: 6 |
| ZmCO-like | Zea mays | Polynucleotide | SEQ ID NO: 7 |
| ZmCCA1 genomic | Zea mays | Polynucleotide | SEQ ID NO: 8 |
| ZmCCA1Exon 1 | Zea mays | Polynucleotide | SEQ ID NO: 9 |
| ZmCCA1Exon 2 | Zea mays | Polynucleotide | SEQ ID NO: 10 |
| ZmCCA1Exon 3 | Zea mays | Polynucleotide | SEQ ID NO: 11 |
| ZmCCA1Exon 4 | Zea mays | Polynucleotide | SEQ ID NO: 12 |
| ZmCCA1Exon 5 | Zea mays | Polynucleotide | SEQ ID NO: 13 |
| ZmCCA1Exon 6 | Zea mays | Polynucleotide | SEQ ID NO: 14 |
| ZmCCA1Exon 7 | Zea mays | Polynucleotide | SEQ ID NO: 15 |
| ZmCCA1Exon 8 | Zea mays | Polynucleotide | SEQ ID NO: 16 |
| ZmCCA1Exon 9 | Zea mays | Polynucleotide | SEQ ID NO: 17 |
| ZmCCA1Exon 10 | Zea mays | Polynucleotide | SEQ ID NO: 18 |
| ZmCCA1Exon 11 | Zea mays | Polynucleotide | SEQ ID NO: 19 |
| ZmLHY genomic | Zea mays | Polynucleotide | SEQ ID NO: 20 |
| ZmLHY Exon 1 | Zea mays | Polynucleotide | SEQ ID NO: 21 |
| ZmLHY Exon 2 | Zea mays | Polynucleotide | SEQ ID NO: 22 |
| ZmLHY Exon 3 | Zea mays | Polynucleotide | SEQ ID NO: 23 |
| ZmLHY Exon 4 | Zea mays | Polynucleotide | SEQ ID NO: 24 |
| ZmLHY Exon 5 | Zea mays | Polynucleotide | SEQ ID NO: 25 |
| ZmLHY Exon 6 | Zea mays | Polynucleotide | SEQ ID NO: 26 |
| ZmLHY Exon 7 | Zea mays | Polynucleotide | SEQ ID NO: 27 |
| ZmLHY Exon 8 | Zea mays | Polynucleotide | SEQ ID NO: 28 |
| ZmLHY Exon 9 | Zea mays | Polynucleotide | SEQ ID NO: 29 |
| ZmLHY Exon 10 | Zea mays | Polynucleotide | SEQ ID NO: 30 |
| Diurnal Promoter #1 | Zea mays | Polynucleotide | SEQ ID NO: 31 |
| Diurnal Promoter #2 | Zea mays | Polynucleotide | SEQ ID NO: 32 |
| Diurnal Promoter #3 | Zea mays | Polynucleotide | SEQ ID NO: 33 |
| Dirunal promoter #4 | Zea mays | Polynucleotide | SEQ ID NO: 34 |
| ZmCCA1 promoter | Zea mays | Polynucleotide | SEQ ID NO: 35 |
| ZmLHY promoter | Zea mays | Polynucleotide | SEQ ID NO: 36 |
| Diurnal promoter#7 | Zea mays | Polynucleotide | SEQ ID NO: 37 |
| Diurnal Promoter #8 | Zea mays | Polynucleotide | SEQ ID NO: 38 |
| Diurnal Promoter #9 | Zea mays | Polynucleotide | SEQ ID NO: 39 |
| ZmTOCa Promoter | Zea mays | Polynucleotide | SEQ ID NO: 40 |
| Diurnal Ear Promoter 1 | Zea mays | Polynucleotide | SEQ ID NO: 41 |
| Diurnal Ear Promoter 2 | Zea mays | Polynucleotide | SEQ ID NO: 42 |
| Diurnal Ear Promoter 3 | Zea mays | Polynucleotide | SEQ ID NO: 43 |
| Diurnal Ear Promoter 4 | Zea mays | Polynucleotide | SEQ ID NO: 44 |
| Diurnal Ear Promoter 5 | Zea mays | Polynucleotide | SEQ ID NO: 45 |
| Diurnal Ear Promoter 7 | Zea mays | Polynucleotide | SEQ ID NO: 46 |
| Diurnal Ear Promoter 8 | Zea mays | Polynucleotide | SEQ ID NO: 47 |
| Diurnal Ear Promoter 9 | Zea mays | Polynucleotide | SEQ ID NO: 48 |
| Diurnal Ear Promoter 10 | Zea mays | Polynucleotide | SEQ ID NO: 49 |
| Diurnal Ear Promoter 11 | Zea mays | Polynucleotide | SEQ ID NO: 50 |
| Diurnal Ear Promoter 12 | Zea mays | Polynucleotide | SEQ ID NO: 51 |
| Diurnal Ear Promoter 13 | Zea mays | Polynucleotide | SEQ ID NO: 52 |
| Diurnal Ear Promoter 14 | Zea mays | Polynucleotide | SEQ ID NO: 53 |
| Diurnal Ear Promoter 15 | Zea mays | Polynucleotide | SEQ ID NO: 54 |
| Diurnal Ear Promoter 16 | Zea mays | Polynucleotide | SEQ ID NO: 55 |
| Diurnal NUE Promoter 1 | Zea mays | Polynucleotide | SEQ ID NO: 56 |
| Diurnal NUE Promoter 2 | Zea mays | Polynucleotide | SEQ ID NO: 57 |
| Diurnal NUE Promoter 3 | Zea mays | Polynucleotide | SEQ ID NO: 58 |
| Diurnal NUE Promoter 4 | Zea mays | Polynucleotide | SEQ ID NO: 59 |
| Diurnal NUE Promoter 5 | Zea mays | Polynucleotide | SEQ ID NO: 60 |
| Diurnal NUE Promoter 6 | Zea mays | Polynucleotide | SEQ ID NO: 61 |
| Diurnal NUE Promoter 7 | Zea mays | Polynucleotide | SEQ ID NO: 62 |
| Diurnal NUE Promoter 8 | Zea mays | Polynucleotide | SEQ ID NO: 63 |
| Diurnal NUE Promoter 9 | Zea mays | Polynucleotide | SEQ ID NO: 64 |
| Diurnal NUE Promoter 10 | Zea mays | Polynucleotide | SEQ ID NO: 65 |
| Diurnal NUE Promoter 11 | Zea mays | Polynucleotide | SEQ ID NO: 66 |
| Diurnal NUE Promoter 12 | Zea mays | Polynucleotide | SEQ ID NO: 67 |
| Diurnal NUE Promoter 13 | Zea mays | Polynucleotide | SEQ ID NO: 68 |
| Diurnal NUE Promoter 14 | Zea mays | Polynucleotide | SEQ ID NO: 69 |
| Diurnal NUE Promoter 15 | Zea mays | Polynucleotide | SEQ ID NO: 70 |
| Diurnal NUE Promoter 16 | Zea mays | Polynucleotide | SEQ ID NO: 71 |
| Diurnal NUE Promoter 17 | Zea mays | Polynucleotide | SEQ ID NO: 72 |
| Diurnal NUE Promoter 18 | Zea mays | Polynucleotide | SEQ ID NO: 73 |
| Diurnal NUE Promoter 19 | Zea mays | Polynucleotide | SEQ ID NO: 74 |
| Diurnal NUE Promoter 20 | Zea mays | Polynucleotide | SEQ ID NO: 75 |
| Diurnal NUE Promoter 21 | Zea mays | Polynucleotide | SEQ ID NO: 76 |
| Diurnal NUE Promoter 22 | Zea mays | Polynucleotide | SEQ ID NO: 77 |
| Diurnal NUE Promoter 23 | Zea mays | Polynucleotide | SEQ ID NO: 78 |
| Diurnal NUE Promoter 24 | Zea mays | Polynucleotide | SEQ ID NO: 79 |
| Diurnal NUE Promoter 25 | Zea mays | Polynucleotide | SEQ ID NO: 80 |
| Diurnal NUE Promoter 26 | Zea mays | Polynucleotide | SEQ ID NO: 81 |
| Diurnal NUE Promoter 27 | Zea mays | Polynucleotide | SEQ ID NO: 82 |
| Diurnal NUE Promoter 28 | Zea mays | Polynucleotide | SEQ ID NO: 83 |
| Diurnal NUE Promoter 29 | Zea mays | Polynucleotide | SEQ ID NO: 84 |
| Diurnal NUE Promoter 30 | Zea mays | Polynucleotide | SEQ ID NO: 85 |
| Diurnal NUE Promoter 31 | Zea mays | Polynucleotide | SEQ ID NO: 86 |
| Diurnal NUE Promoter 32 | Zea mays | Polynucleotide | SEQ ID NO: 87 |
| Diurnal NUE Promoter 33 | Zea mays | Polynucleotide | SEQ ID NO: 88 |
| Diurnal NUE Promoter 34 | Zea mays | Polynucleotide | SEQ ID NO: 89 |
| Diurnal NUE Promoter 35 | Zea mays | Polynucleotide | SEQ ID NO: 90 |
| Diurnal NUE Promoter 36 | Zea mays | Polynucleotide | SEQ ID NO: 91 |
| Diurnal NUE Promoter 37 | Zea mays | Polynucleotide | SEQ ID NO: 92 |
| Diurnal NUE Promoter 38 | Zea mays | Polynucleotide | SEQ ID NO: 93 |
| Diurnal NUE Promoter 39 | Zea mays | Polynucleotide | SEQ ID NO: 94 |
| Diurnal NUE Promoter 40 | Zea mays | Polynucleotide | SEQ ID NO: 95 |
| Diurnal NUE Promoter 41 | Zea mays | Polynucleotide | SEQ ID NO: 96 |
| Diurnal NUE Promoter 42 | Zea mays | Polynucleotide | SEQ ID NO: 97 |
| Diurnal NUE Promoter 43 | Zea mays | Polynucleotide | SEQ ID NO: 98 |
| Diurnal NUE Promoter 44 | Zea mays | Polynucleotide | SEQ ID NO: 99 |
| Diurnal NUE Promoter 45 | Zea mays | Polynucleotide | SEQ ID NO: 100 |
| Diurnal NUE Promoter 46 | Zea mays | Polynucleotide | SEQ ID NO: 101 |
| Diurnal NUE Promoter 47 | Zea mays | Polynucleotide | SEQ ID NO: 102 |
| Diurnal NUE Promoter 48 | Zea mays | Polynucleotide | SEQ ID NO: 103 |
| Diurnal NUE Promoter 49 | Zea mays | Polynucleotide | SEQ ID NO: 104 |
| Diurnal NUE Promoter 50 | Zea mays | Polynucleotide | SEQ ID NO: 105 |
| Diurnal NUE Promoter 51 | Zea mays | Polynucleotide | SEQ ID NO: 106 |
| Diurnal NUE Promoter 52 | Zea mays | Polynucleotide | SEQ ID NO: 107 |
| Diurnal NUE Promoter 53 | Zea mays | Polynucleotide | SEQ ID NO: 108 |
| Diurnal NUE Promoter 54 | Zea mays | Polynucleotide | SEQ ID NO: 109 |
| Diurnal NUE Promoter 55 | Zea mays | Polynucleotide | SEQ ID NO: 110 |
| Diurnal NUE Promoter 56 | Zea mays | Polynucleotide | SEQ ID NO: 111 |
| Diurnal NUE Promoter 57 | Zea mays | Polynucleotide | SEQ ID NO: 112 |
| Diurnal NUE Promoter 58 | Zea mays | Polynucleotide | SEQ ID NO: 113 |
| Diurnal NUE Promoter 59 | Zea mays | Polynucleotide | SEQ ID NO: 114 |
| Diurnal NUE Promoter 60 | Zea mays | Polynucleotide | SEQ ID NO: 115 |
| Diurnal NUE Promoter 61 | Zea mays | Polynucleotide | SEQ ID NO: 116 |
| Diurnal AMP Promoter 1 | Zea mays | Polynucleotide | SEQ ID NO: 117 |
| Diurnal AMP Promoter 2 | Zea mays | Polynucleotide | SEQ ID NO: 118 |
| Diurnal AMP Promoter 3 | Zea mays | Polynucleotide | SEQ ID NO: 119 |
| Diurnal AMP Promoter 4 | Zea mays | Polynucleotide | SEQ ID NO: 120 |
| Diurnal AMP Promoter 5 | Zea mays | Polynucleotide | SEQ ID NO: 121 |
| Diurnal AMP Promoter 6 | Zea mays | Polynucleotide | SEQ ID NO: 122 |
| Diurnal AMP Promoter 7 | Zea mays | Polynucleotide | SEQ ID NO: 123 |
| Diurnal AMP Promoter 8 | Zea mays | Polynucleotide | SEQ ID NO: 124 |
| Diurnal AMP Promoter 9 | Zea mays | Polynucleotide | SEQ ID NO: 125 |
| Diurnal AMP Promoter 10 | Zea mays | Polynucleotide | SEQ ID NO: 126 |
| Diurnal AMP Promoter 11 | Zea mays | Polynucleotide | SEQ ID NO: 127 |
| Diurnal AMP Promoter 12 | Zea mays | Polynucleotide | SEQ ID NO: 128 |
| Diurnal AMP Promoter 13 | Zea mays | Polynucleotide | SEQ ID NO: 129 |
| Diurnal AMP Promoter 14 | Zea mays | Polynucleotide | SEQ ID NO: 130 |
| Diurnal AMP Promoter 15 | Zea mays | Polynucleotide | SEQ ID NO: 131 |
| Diurnal AMP Promoter 16 | Zea mays | Polynucleotide | SEQ ID NO: 132 |
| Diurnal AMP Promoter 17 | Zea mays | Polynucleotide | SEQ ID NO: 133 |
| Diurnal AMP Promoter 18 | Zea mays | Polynucleotide | SEQ ID NO: 134 |
| Diurnal AMP Promoter 19 | Zea mays | Polynucleotide | SEQ ID NO: 135 |
| Diurnal AMP Promoter 20 | Zea mays | Polynucleotide | SEQ ID NO: 136 |
| Diurnal AMP Promoter 21 | Zea mays | Polynucleotide | SEQ ID NO: 137 |
| Diurnal AMP Promoter 22 | Zea mays | Polynucleotide | SEQ ID NO: 138 |
| Diurnal AMP Promoter 23 | Zea mays | Polynucleotide | SEQ ID NO: 139 |
| Diurnal AMP Promoter 24 | Zea mays | Polynucleotide | SEQ ID NO: 140 |
| Diurnal AMP Promoter 25 | Zea mays | Polynucleotide | SEQ ID NO: 141 |
| Diurnal AMP Promoter 26 | Zea mays | Polynucleotide | SEQ ID NO: 142 |
| Diurnal AMP Promoter 27 | Zea mays | Polynucleotide | SEQ ID NO: 143 |
| Diurnal AMP Promoter 28 | Zea mays | Polynucleotide | SEQ ID NO: 144 |
| Diurnal AMP Promoter 29 | Zea mays | Polynucleotide | SEQ ID NO: 145 |
| Diurnal AMP Promoter 30 | Zea mays | Polynucleotide | SEQ ID NO: 146 |
| Diurnal AMP Promoter 31 | Zea mays | Polynucleotide | SEQ ID NO: 147 |
| Diurnal AMP Promoter 32 | Zea mays | Polynucleotide | SEQ ID NO: 148 |
| Diurnal AMP Promoter 33 | Zea mays | Polynucleotide | SEQ ID NO: 149 |
| Diurnal AMP Promoter 34 | Zea mays | Polynucleotide | SEQ ID NO: 150 |
| Diurnal AMP Promoter 35 | Zea mays | Polynucleotide | SEQ ID NO: 151 |
| Diurnal AMP Promoter 36 | Zea mays | Polynucleotide | SEQ ID NO: 152 |
| Diurnal AMP Promoter 37 | Zea mays | Polynucleotide | SEQ ID NO: 153 |
| Diurnal AMP Promoter 38 | Zea mays | Polynucleotide | SEQ ID NO: 154 |
| Diurnal AMP Promoter 39 | Zea mays | Polynucleotide | SEQ ID NO: 155 |
| Diurnal AMP Promoter 40 | Zea mays | Polynucleotide | SEQ ID NO: 156 |
| Diurnal AMP Promoter 41 | Zea mays | Polynucleotide | SEQ ID NO: 157 |
| Diurnal AMP Promoter 42 | Zea mays | Polynucleotide | SEQ ID NO: 158 |
| Diurnal AMP Promoter 43 | Zea mays | Polynucleotide | SEQ ID NO: 159 |
| Diurnal AMP Promoter 44 | Zea mays | Polynucleotide | SEQ ID NO: 160 |
| Diurnal AMP Promoter 45 | Zea mays | Polynucleotide | SEQ ID NO: 161 |
| Diurnal AMP Promoter 46 | Zea mays | Polynucleotide | SEQ ID NO: 162 |
| Diurnal AMP Promoter 47 | Zea mays | Polynucleotide | SEQ ID NO: 163 |
| Diurnal AMP Promoter 48 | Zea mays | Polynucleotide | SEQ ID NO: 164 |
| Diurnal AMP Promoter 49 | Zea mays | Polynucleotide | SEQ ID NO: 165 |
| Diurnal AMP Promoter 50 | Zea mays | Polynucleotide | SEQ ID NO: 166 |
| Diurnal AMP Promoter 51 | Zea mays | Polynucleotide | SEQ ID NO: 167 |
| Diurnal AMP Promoter 52 | Zea mays | Polynucleotide | SEQ ID NO: 168 |
| Diurnal AMP Promoter 53 | Zea mays | Polynucleotide | SEQ ID NO: 169 |
| Diurnal AMP Promoter 54 | Zea mays | Polynucleotide | SEQ ID NO: 170 |
| Diurnal AMP Promoter 55 | Zea mays | Polynucleotide | SEQ ID NO: 171 |
| Diurnal AMP Promoter 56 | Zea mays | Polynucleotide | SEQ ID NO: 172 |
| Diurnal AMP Promoter 57 | Zea mays | Polynucleotide | SEQ ID NO: 173 |
| Diurnal AMP Promoter 58 | Zea mays | Polynucleotide | SEQ ID NO: 174 |
| Diurnal AMP Promoter 59 | Zea mays | Polynucleotide | SEQ ID NO: 175 |
| Diurnal AMP Promoter 60 | Zea mays | Polynucleotide | SEQ ID NO: 176 |
| Diurnal AMP Promoter 61 | Zea mays | Polynucleotide | SEQ ID NO: 177 |
| Diurnal AMP Promoter 62 | Zea mays | Polynucleotide | SEQ ID NO: 178 |
| Diurnal AMP Promoter 63 | Zea mays | Polynucleotide | SEQ ID NO: 179 |
| Diurnal AMP Promoter 64 | Zea mays | Polynucleotide | SEQ ID NO: 180 |
| Diurnal AMP Promoter 65 | Zea mays | Polynucleotide | SEQ ID NO: 181 |
| Diurnal AMP Promoter 66 | Zea mays | Polynucleotide | SEQ ID NO: 182 |
| Diurnal AMP Promoter 67 | Zea mays | Polynucleotide | SEQ ID NO: 183 |
| Diurnal Ear 1 | Zea mays | Polynucleotide | SEQ ID NO: 184 |
| Diurnal Ear 1 | Zea mays | Polypeptide | SEQ ID NO: 185 |
| Diurnal Ear 2 | Zea mays | Polynucleotide | SEQ ID NO: 186 |
| Diurnal Ear 2 | Zea mays | Polypeptide | SEQ ID NO: 187 |
| Diurnal Ear 3 | Zea mays | Polynucleotide | SEQ ID NO: 188 |
| Diurnal Ear 3 | Zea mays | Polypeptide | SEQ ID NO: 189 |
| Diurnal Ear 4 | Zea mays | Polynucleotide | SEQ ID NO: 190 |
| Diurnal Ear 4 | Zea mays | Polypeptide | SEQ ID NO: 191 |
| Diurnal Ear 5 | Zea mays | Polynucleotide | SEQ ID NO: 192 |
| Diurnal Ear 5 | Zea mays | Polypeptide | SEQ ID NO: 193 |
| Diurnal Ear 6 | Zea mays | Polynucleotide | SEQ ID NO: 194 |
| Diurnal Ear 6 | Zea mays | Polypeptide | SEQ ID NO: 195 |
| Diurnal Ear 7 | Zea mays | Polynucleotide | SEQ ID NO: 196 |
| Diurnal Ear 7 | Zea mays | Polypeptide | SEQ ID NO: 197 |
| Diurnal Ear 8 | Zea mays | Polynucleotide | SEQ ID NO: 198 |
| Diurnal Ear 8 | Zea mays | Polypeptide | SEQ ID NO: 199 |
| Diurnal Ear 9 | Zea mays | Polynucleotide | SEQ ID NO: 200 |
| Diurnal Ear 9 | Zea mays | Polypeptide | SEQ ID NO: 201 |
| Diurnal Ear 10 | Zea mays | Polynucleotide | SEQ ID NO: 202 |
| Diurnal Ear 10 | Zea mays | Polypeptide | SEQ ID NO: 203 |
| Diurnal Ear 11 | Zea mays | Polynucleotide | SEQ ID NO: 204 |
| Diurnal Ear 11 | Zea mays | Polypeptide | SEQ ID NO: 205 |
| Diurnal Ear 12 | Zea mays | Polynucleotide | SEQ ID NO: 206 |
| Diurnal Ear 12 | Zea mays | Polypeptide | SEQ ID NO: 207 |
| Diurnal Ear 13 | Zea mays | Polynucleotide | SEQ ID NO: 208 |
| Diurnal Ear 13 | Zea mays | Polypeptide | SEQ ID NO: 209 |
| Diurnal Ear 14 | Zea mays | Polynucleotide | SEQ ID NO: 210 |
| Diurnal Ear 14 | Zea mays | Polypeptide | SEQ ID NO: 211 |
| Diurnal Ear 15 | Zea mays | Polynucleotide | SEQ ID NO: 212 |
| Diurnal Ear 15 | Zea mays | Polypeptide | SEQ ID NO: 213 |
| Diurnal Ear 16 | Zea mays | Polynucleotide | SEQ ID NO: 214 |
| Diurnal Ear 16 | Zea mays | Polypeptide | SEQ ID NO: 215 |
| Diurnal NUE 1 | Zea mays | Polynucleotide | SEQ ID NO: 216 |
| Diurnal NUE 1 | Zea mays | Polypeptide | SEQ ID NO: 217 |
| Diurnal NUE 2 | Zea mays | Polynucleotide | SEQ ID NO: 218 |
| Diurnal NUE 2 | Zea mays | Polypeptide | SEQ ID NO: 219 |
| Diurnal NUE 3 | Zea mays | Polynucleotide | SEQ ID NO: 220 |
| Diurnal NUE 3 | Zea mays | Polypeptide | SEQ ID NO: 221 |
| Diurnal NUE 4 | Zea mays | Polynucleotide | SEQ ID NO: 222 |
| Diurnal NUE 4 | Zea mays | Polypeptide | SEQ ID NO: 223 |
| Diurnal NUE 5 | Zea mays | Polynucleotide | SEQ ID NO: 224 |
| Diurnal NUE 5 | Zea mays | Polypeptide | SEQ ID NO: 225 |
| Diurnal NUE 6 | Zea mays | Polynucleotide | SEQ ID NO: 226 |
| Diurnal NUE 6 | Zea mays | Polypeptide | SEQ ID NO: 227 |
| Diurnal NUE 7 | Zea mays | Polynucleotide | SEQ ID NO: 228 |
| Diurnal NUE 7 | Zea mays | Polypeptide | SEQ ID NO: 229 |
| Diurnal NUE 8 | Zea mays | Polynucleotide | SEQ ID NO: 230 |
| Diurnal NUE 8 | Zea mays | Polypeptide | SEQ ID NO: 231 |
| Diurnal NUE 9 | Zea mays | Polynucleotide | SEQ ID NO: 232 |
| Diurnal NUE 9 | Zea mays | Polypeptide | SEQ ID NO: 233 |
| Diurnal NUE 10 | Zea mays | Polynucleotide | SEQ ID NO: 234 |
| Diurnal NUE 10 | Zea mays | Polypeptide | SEQ ID NO: 235 |
| Diurnal NUE 11 | Zea mays | Polynucleotide | SEQ ID NO: 236 |
| Diurnal NUE 11 | Zea mays | Polypeptide | SEQ ID NO: 237 |
| Diurnal NUE 12 | Zea mays | Polynucleotide | SEQ ID NO: 238 |
| Diurnal NUE 12 | Zea mays | Polypeptide | SEQ ID NO: 239 |
| Diurnal NUE 13 | Zea mays | Polynucleotide | SEQ ID NO: 240 |
| Diurnal NUE 13 | Zea mays | Polypeptide | SEQ ID NO: 241 |
| Diurnal NUE 14 | Zea mays | Polynucleotide | SEQ ID NO: 242 |
| Diurnal NUE 14 | Zea mays | Polypeptide | SEQ ID NO: 243 |
| Diurnal NUE 15 | Zea mays | Polynucleotide | SEQ ID NO: 244 |
| Diurnal NUE 15 | Zea mays | Polypeptide | SEQ ID NO: 245 |
| Diurnal NUE 16 | Zea mays | Polynucleotide | SEQ ID NO: 246 |
| Diurnal NUE 16 | Zea mays | Polypeptide | SEQ ID NO: 247 |
| Diurnal NUE 17 | Zea mays | Polynucleotide | SEQ ID NO: 248 |
| Diurnal NUE 17 | Zea mays | Polypeptide | SEQ ID NO: 249 |
| Diurnal NUE 18 | Zea mays | Polynucleotide | SEQ ID NO: 250 |
| Diurnal NUE 18 | Zea mays | Polypeptide | SEQ ID NO: 251 |
| Diurnal NUE 19 | Zea mays | Polynucleotide | SEQ ID NO: 252 |
| Diurnal NUE 19 | Zea mays | Polypeptide | SEQ ID NO: 253 |
| Diurnal NUE 20 | Zea mays | Polynucleotide | SEQ ID NO: 254 |
| Diurnal NUE 20 | Zea mays | Polypeptide | SEQ ID NO: 255 |
| Diurnal NUE 21 | Zea mays | Polynucleotide | SEQ ID NO: 256 |
| Diurnal NUE 21 | Zea mays | Polypeptide | SEQ ID NO: 257 |
| Diurnal NUE 22 | Zea mays | Polynucleotide | SEQ ID NO: 258 |
| Diurnal NUE 22 | Zea mays | Polypeptide | SEQ ID NO: 259 |
| Diurnal NUE 23 | Zea mays | Polynucleotide | SEQ ID NO: 260 |
| Diurnal NUE 23 | Zea mays | Polypeptide | SEQ ID NO: 261 |
| Diurnal NUE 24 | Zea mays | Polynucleotide | SEQ ID NO: 262 |
| Diurnal NUE 24 | Zea mays | Polypeptide | SEQ ID NO: 263 |
| Diurnal NUE 25 | Zea mays | Polynucleotide | SEQ ID NO: 264 |
| Diurnal NUE 25 | Zea mays | Polypeptide | SEQ ID NO: 265 |
| Diurnal NUE 26 | Zea mays | Polynucleotide | SEQ ID NO: 266 |
| Diurnal NUE 26 | Zea mays | Polypeptide | SEQ ID NO: 267 |
| Diurnal NUE 27 | Zea mays | Polynucleotide | SEQ ID NO: 268 |
| Diurnal NUE 27 | Zea mays | Polypeptide | SEQ ID NO: 269 |
| Diurnal NUE 28 | Zea mays | Polynucleotide | SEQ ID NO: 270 |
| Diurnal NUE 28 | Zea mays | Polypeptide | SEQ ID NO: 271 |
| Diurnal NUE 29 | Zea mays | Polynucleotide | SEQ ID NO: 272 |
| Diurnal NUE 29 | Zea mays | Polypeptide | SEQ ID NO: 273 |
| Diurnal NUE 30 | Zea mays | Polynucleotide | SEQ ID NO: 274 |
| Diurnal NUE 30 | Zea mays | Polypeptide | SEQ ID NO: 275 |
| Diurnal NUE 31 | Zea mays | Polynucleotide | SEQ ID NO: 276 |
| Diurnal NUE 31 | Zea mays | Polypeptide | SEQ ID NO: 277 |
| Diurnal NUE 32 | Zea mays | Polynucleotide | SEQ ID NO: 278 |
| Diurnal NUE 32 | Zea mays | Polypeptide | SEQ ID NO: 279 |
| Diurnal NUE 33 | Zea mays | Polynucleotide | SEQ ID NO: 280 |
| Diurnal NUE 33 | Zea mays | Polypeptide | SEQ ID NO: 281 |
| Diurnal NUE 34 | Zea mays | Polynucleotide | SEQ ID NO: 282 |
| Diurnal NUE 34 | Zea mays | Polypeptide | SEQ ID NO: 283 |
| Diurnal NUE 35 | Zea mays | Polynucleotide | SEQ ID NO: 284 |
| Diurnal NUE 35 | Zea mays | Polypeptide | SEQ ID NO: 285 |
| Diurnal NUE 36 | Zea mays | Polynucleotide | SEQ ID NO: 286 |
| Diurnal NUE 36 | Zea mays | Polypeptide | SEQ ID NO: 287 |
| Diurnal NUE 37 | Zea mays | Polynucleotide | SEQ ID NO: 288 |
| Diurnal NUE 37 | Zea mays | Polypeptide | SEQ ID NO: 289 |
| Diurnal NUE 38 | Zea mays | Polynucleotide | SEQ ID NO: 290 |
| Diurnal NUE 38 | Zea mays | Polypeptide | SEQ ID NO: 291 |
| Diurnal NUE 39 | Zea mays | Polynucleotide | SEQ ID NO: 292 |
| Diurnal NUE 39 | Zea mays | Polypeptide | SEQ ID NO: 293 |
| Diurnal NUE 40 | Zea mays | Polynucleotide | SEQ ID NO: 294 |
| Diurnal NUE 40 | Zea mays | Polypeptide | SEQ ID NO: 295 |
| Diurnal NUE 41 | Zea mays | Polynucleotide | SEQ ID NO: 296 |
| Diurnal NUE 41 | Zea mays | Polypeptide | SEQ ID NO: 297 |
| Diurnal NUE 42 | Zea mays | Polynucleotide | SEQ ID NO: 298 |
| Diurnal NUE 42 | Zea mays | Polypeptide | SEQ ID NO: 299 |
| Diurnal NUE 43 | Zea mays | Polynucleotide | SEQ ID NO: 300 |
| Diurnal NUE 43 | Zea mays | Polypeptide | SEQ ID NO: 301 |
| Diurnal NUE 44 | Zea mays | Polynucleotide | SEQ ID NO: 302 |
| Diurnal NUE 44 | Zea mays | Polypeptide | SEQ ID NO: 303 |
| Diurnal NUE 45 | Zea mays | Polynucleotide | SEQ ID NO: 304 |
| Diurnal NUE 45 | Zea mays | Polypeptide | SEQ ID NO: 305 |
| Diurnal NUE 46 | Zea mays | Polynucleotide | SEQ ID NO: 306 |
| Diurnal NUE 46 | Zea mays | Polypeptide | SEQ ID NO: 307 |
| Diurnal NUE 47 | Zea mays | Polynucleotide | SEQ ID NO: 308 |
| Diurnal NUE 47 | Zea mays | Polypeptide | SEQ ID NO: 309 |
| Diurnal NUE 48 | Zea mays | Polynucleotide | SEQ ID NO: 310 |
| Diurnal NUE 48 | Zea mays | Polypeptide | SEQ ID NO: 311 |
| Diurnal NUE 49 | Zea mays | Polynucleotide | SEQ ID NO: 312 |
| Diurnal NUE 49 | Zea mays | Polypeptide | SEQ ID NO: 313 |
| Diurnal NUE 50 | Zea mays | Polynucleotide | SEQ ID NO: 314 |
| Diurnal NUE 50 | Zea mays | Polypeptide | SEQ ID NO: 315 |
| Diurnal NUE 51 | Zea mays | Polynucleotide | SEQ ID NO: 316 |
| Diurnal NUE 51 | Zea mays | Polypeptide | SEQ ID NO: 317 |
| Diurnal NUE 52 | Zea mays | Polynucleotide | SEQ ID NO: 318 |
| Diurnal NUE 52 | Zea mays | Polypeptide | SEQ ID NO: 319 |
| Diurnal NUE 53 | Zea mays | Polynucleotide | SEQ ID NO: 320 |
| Diurnal NUE 53 | Zea mays | Polypeptide | SEQ ID NO: 321 |
| Diurnal NUE 54 | Zea mays | Polynucleotide | SEQ ID NO: 322 |
| Diurnal NUE 54 | Zea mays | Polypeptide | SEQ ID NO: 323 |
| Diurnal NUE 55 | Zea mays | Polynucleotide | SEQ ID NO: 324 |
| Diurnal NUE 55 | Zea mays | Polypeptide | SEQ ID NO: 325 |
| Diurnal NUE 56 | Zea mays | Polynucleotide | SEQ ID NO: 326 |
| Diurnal NUE 56 | Zea mays | Polypeptide | SEQ ID NO: 327 |
| Diurnal NUE 57 | Zea mays | Polynucleotide | SEQ ID NO: 328 |
| Diurnal NUE 57 | Zea mays | Polypeptide | SEQ ID NO: 329 |
| Diurnal NUE 58 | Zea mays | Polynucleotide | SEQ ID NO: 330 |
| Diurnal NUE 58 | Zea mays | Polypeptide | SEQ ID NO: 331 |
| Diurnal NUE 59 | Zea mays | Polynucleotide | SEQ ID NO: 332 |
| Diurnal NUE 59 | Zea mays | Polypeptide | SEQ ID NO: 333 |
| Diurnal NUE 60 | Zea mays | Polynucleotide | SEQ ID NO: 334 |
| Diurnal NUE 60 | Zea mays | Polypeptide | SEQ ID NO: 335 |
| Diurnal NUE 61 | Zea mays | Polynucleotide | SEQ ID NO: 336 |
| Diurnal NUE 61 | Zea mays | Polypeptide | SEQ ID NO: 337 |
| Diurnal AMP 1 | Zea mays | Polynucleotide | SEQ ID NO: 338 |
| Diurnal AMP 1 | Zea mays | Polypeptide | SEQ ID NO: 339 |
| Diurnal AMP 2 | Zea mays | Polynucleotide | SEQ ID NO: 340 |
| Diurnal AMP 2 | Zea mays | Polypeptide | SEQ ID NO: 341 |
| Diurnal AMP 3 | Zea mays | Polynucleotide | SEQ ID NO: 342 |
| Diurnal AMP 3 | Zea mays | Polypeptide | SEQ ID NO: 343 |
| Diurnal AMP 4 | Zea mays | Polynucleotide | SEQ ID NO: 344 |
| Diurnal AMP 4 | Zea mays | Polypeptide | SEQ ID NO: 345 |
| Diurnal AMP 5 | Zea mays | Polynucleotide | SEQ ID NO: 346 |
| Diurnal AMP 5 | Zea mays | Polypeptide | SEQ ID NO: 347 |
| Diurnal AMP 6 | Zea mays | Polynucleotide | SEQ ID NO: 348 |
| Diurnal AMP 6 | Zea mays | Polypeptide | SEQ ID NO: 349 |
| Diurnal AMP 7 | Zea mays | Polynucleotide | SEQ ID NO: 350 |
| Diurnal AMP 7 | Zea mays | Polypeptide | SEQ ID NO: 351 |
| Diurnal AMP 8 | Zea mays | Polynucleotide | SEQ ID NO: 352 |
| Diurnal AMP 8 | Zea mays | Polypeptide | SEQ ID NO: 353 |
| Diurnal AMP 9 | Zea mays | Polynucleotide | SEQ ID NO: 354 |
| Diurnal AMP 9 | Zea mays | Polypeptide | SEQ ID NO: 355 |
| Diurnal AMP 10 | Zea mays | Polynucleotide | SEQ ID NO: 356 |
| Diurnal AMP 10 | Zea mays | Polypeptide | SEQ ID NO: 357 |
| Diurnal AMP 11 | Zea mays | Polynucleotide | SEQ ID NO: 358 |
| Diurnal AMP 11 | Zea mays | Polypeptide | SEQ ID NO: 359 |
| Diurnal AMP 12 | Zea mays | Polynucleotide | SEQ ID NO: 360 |
| Diurnal AMP 12 | Zea mays | Polypeptide | SEQ ID NO: 361 |
| Diurnal AMP 13 | Zea mays | Polynucleotide | SEQ ID NO: 362 |
| Diurnal AMP 13 | Zea mays | Polypeptide | SEQ ID NO: 363 |
| Diurnal AMP 14 | Zea mays | Polynucleotide | SEQ ID NO: 364 |
| Diurnal AMP 14 | Zea mays | Polypeptide | SEQ ID NO: 365 |
| Diurnal AMP 15 | Zea mays | Polynucleotide | SEQ ID NO: 366 |
| Diurnal AMP 15 | Zea mays | Polypeptide | SEQ ID NO: 367 |
| Diurnal AMP 16 | Zea mays | Polynucleotide | SEQ ID NO: 368 |
| Diurnal AMP 16 | Zea mays | Polypeptide | SEQ ID NO: 369 |
| Diurnal AMP 17 | Zea mays | Polynucleotide | SEQ ID NO: 370 |
| Diurnal AMP 17 | Zea mays | Polypeptide | SEQ ID NO: 371 |
| Diurnal AMP 18 | Zea mays | Polynucleotide | SEQ ID NO: 372 |
| Diurnal AMP 18 | Zea mays | Polypeptide | SEQ ID NO: 373 |
| Diurnal AMP 19 | Zea mays | Polynucleotide | SEQ ID NO: 374 |
| Diurnal AMP 19 | Zea mays | Polypeptide | SEQ ID NO: 375 |
| Diurnal AMP 20 | Zea mays | Polynucleotide | SEQ ID NO: 376 |
| Diurnal AMP 20 | Zea mays | Polypeptide | SEQ ID NO: 377 |
| Diurnal AMP 21 | Zea mays | Polynucleotide | SEQ ID NO: 378 |
| Diurnal AMP 21 | Zea mays | Polypeptide | SEQ ID NO: 379 |
| Diurnal AMP 22 | Zea mays | Polynucleotide | SEQ ID NO: 380 |
| Diurnal AMP 22 | Zea mays | Polypeptide | SEQ ID NO: 381 |
| Diurnal AMP 23 | Zea mays | Polynucleotide | SEQ ID NO: 382 |
| Diurnal AMP 23 | Zea mays | Polypeptide | SEQ ID NO: 383 |
| Diurnal AMP 24 | Zea mays | Polynucleotide | SEQ ID NO: 384 |
| Diurnal AMP 24 | Zea mays | Polypeptide | SEQ ID NO: 385 |
| Diurnal AMP 25 | Zea mays | Polynucleotide | SEQ ID NO: 386 |
| Diurnal AMP 25 | Zea mays | Polypeptide | SEQ ID NO: 387 |
| Diurnal AMP 26 | Zea mays | Polynucleotide | SEQ ID NO: 388 |
| Diurnal AMP 26 | Zea mays | Polypeptide | SEQ ID NO: 389 |
| Diurnal AMP 27 | Zea mays | Polynucleotide | SEQ ID NO: 390 |
| Diurnal AMP 27 | Zea mays | Polypeptide | SEQ ID NO: 391 |
| Diurnal AMP 28 | Zea mays | Polynucleotide | SEQ ID NO: 392 |
| Diurnal AMP 28 | Zea mays | Polypeptide | SEQ ID NO: 393 |
| Diurnal AMP 29 | Zea mays | Polynucleotide | SEQ ID NO: 394 |
| Diurnal AMP 29 | Zea mays | Polypeptide | SEQ ID NO: 395 |
| Diurnal AMP 30 | Zea mays | Polynucleotide | SEQ ID NO: 396 |
| Diurnal AMP 30 | Zea mays | Polypeptide | SEQ ID NO: 397 |
| Diurnal AMP 31 | Zea mays | Polynucleotide | SEQ ID NO: 398 |
| Diurnal AMP 31 | Zea mays | Polypeptide | SEQ ID NO: 399 |
| Diurnal AMP 32 | Zea mays | Polynucleotide | SEQ ID NO: 400 |
| Diurnal AMP 32 | Zea mays | Polypeptide | SEQ ID NO: 401 |
| Diurnal AMP 33 | Zea mays | Polynucleotide | SEQ ID NO: 402 |
| Diurnal AMP 33 | Zea mays | Polypeptide | SEQ ID NO: 403 |
| Diurnal AMP 34 | Zea mays | Polynucleotide | SEQ ID NO: 404 |
| Diurnal AMP 34 | Zea mays | Polypeptide | SEQ ID NO: 405 |
| Diurnal AMP 35 | Zea mays | Polynucleotide | SEQ ID NO: 406 |
| Diurnal AMP 35 | Zea mays | Polypeptide | SEQ ID NO: 407 |
| Diurnal AMP 36 | Zea mays | Polynucleotide | SEQ ID NO: 408 |
| Diurnal AMP 36 | Zea mays | Polypeptide | SEQ ID NO: 409 |
| Diurnal AMP 37 | Zea mays | Polynucleotide | SEQ ID NO: 410 |
| Diurnal AMP 37 | Zea mays | Polypeptide | SEQ ID NO: 411 |
| Diurnal AMP 38 | Zea mays | Polynucleotide | SEQ ID NO: 412 |
| Diurnal AMP 38 | Zea mays | Polypeptide | SEQ ID NO: 413 |
| Diurnal AMP 39 | Zea mays | Polynucleotide | SEQ ID NO: 414 |
| Diurnal AMP 39 | Zea mays | Polypeptide | SEQ ID NO: 415 |
| Diurnal AMP 40 | Zea mays | Polynucleotide | SEQ ID NO: 416 |
| Diurnal AMP 40 | Zea mays | Polypeptide | SEQ ID NO: 417 |
| Diurnal AMP 41 | Zea mays | Polynucleotide | SEQ ID NO: 418 |
| Diurnal AMP 41 | Zea mays | Polypeptide | SEQ ID NO: 419 |
| Diurnal AMP 42 | Zea mays | Polynucleotide | SEQ ID NO: 420 |
| Diurnal AMP 42 | Zea mays | Polypeptide | SEQ ID NO: 421 |
| Diurnal AMP 43 | Zea mays | Polynucleotide | SEQ ID NO: 422 |
| Diurnal AMP 43 | Zea mays | Polypeptide | SEQ ID NO: 423 |
| Diurnal AMP 44 | Zea mays | Polynucleotide | SEQ ID NO: 424 |
| Diurnal AMP 44 | Zea mays | Polypeptide | SEQ ID NO: 425 |
| Diurnal AMP 45 | Zea mays | Polynucleotide | SEQ ID NO: 426 |
| Diurnal AMP 45 | Zea mays | Polypeptide | SEQ ID NO: 427 |
| Diurnal AMP 46 | Zea mays | Polynucleotide | SEQ ID NO: 428 |
| Diurnal AMP 46 | Zea mays | Polypeptide | SEQ ID NO: 429 |
| Diurnal AMP 47 | Zea mays | Polynucleotide | SEQ ID NO: 430 |
| Diurnal AMP 47 | Zea mays | Polypeptide | SEQ ID NO: 431 |
| Diurnal AMP 48 | Zea mays | Polynucleotide | SEQ ID NO: 432 |
| Diurnal AMP 48 | Zea mays | Polypeptide | SEQ ID NO: 433 |
| Diurnal AMP 49 | Zea mays | Polynucleotide | SEQ ID NO: 434 |
| Diurnal AMP 49 | Zea mays | Polypeptide | SEQ ID NO: 435 |
| Diurnal AMP 50 | Zea mays | Polynucleotide | SEQ ID NO: 436 |
| Diurnal AMP 50 | Zea mays | Polypeptide | SEQ ID NO: 437 |
| Diurnal AMP 51 | Zea mays | Polynucleotide | SEQ ID NO: 438 |
| Diurnal AMP 51 | Zea mays | Polypeptide | SEQ ID NO: 439 |
| Diurnal AMP 52 | Zea mays | Polynucleotide | SEQ ID NO: 440 |
| Diurnal AMP 52 | Zea mays | Polypeptide | SEQ ID NO: 441 |
| Diurnal AMP 53 | Zea mays | Polynucleotide | SEQ ID NO: 442 |
| Diurnal AMP 53 | Zea mays | Polypeptide | SEQ ID NO: 443 |
| Diurnal AMP 54 | Zea mays | Polynucleotide | SEQ ID NO: 444 |
| Diurnal AMP 54 | Zea mays | Polypeptide | SEQ ID NO: 445 |
| Diurnal AMP 55 | Zea mays | Polynucleotide | SEQ ID NO: 446 |
| Diurnal AMP 55 | Zea mays | Polypeptide | SEQ ID NO: 447 |
| Diurnal AMP 56 | Zea mays | Polynucleotide | SEQ ID NO: 448 |
| Diurnal AMP 56 | Zea mays | Polypeptide | SEQ ID NO: 449 |
| Diurnal AMP 57 | Zea mays | Polynucleotide | SEQ ID NO: 450 |
| Diurnal AMP 57 | Zea mays | Polypeptide | SEQ ID NO: 451 |
| Diurnal AMP 58 | Zea mays | Polynucleotide | SEQ ID NO: 452 |
| Diurnal AMP 58 | Zea mays | Polypeptide | SEQ ID NO: 453 |
| Diurnal AMP 59 | Zea mays | Polynucleotide | SEQ ID NO: 454 |
| Diurnal AMP 59 | Zea mays | Polypeptide | SEQ ID NO: 455 |
| Diurnal AMP 60 | Zea mays | Polynucleotide | SEQ ID NO: 456 |
| Diurnal AMP 60 | Zea mays | Polypeptide | SEQ ID NO: 457 |
| Diurnal AMP 61 | Zea mays | Polynucleotide | SEQ ID NO: 458 |
| Diurnal AMP 61 | Zea mays | Polypeptide | SEQ ID NO: 459 |
| Diurnal AMP 62 | Zea mays | Polynucleotide | SEQ ID NO: 460 |
| Diurnal AMP 62 | Zea mays | Polypeptide | SEQ ID NO: 461 |
| Diurnal AMP 63 | Zea mays | Polynucleotide | SEQ ID NO: 462 |
| Diurnal AMP 63 | Zea mays | Polypeptide | SEQ ID NO: 463 |
| Diurnal AMP 64 | Zea mays | Polynucleotide | SEQ ID NO: 464 |
| Diurnal AMP 64 | Zea mays | Polypeptide | SEQ ID NO: 465 |
| Diurnal AMP 65 | Zea mays | Polynucleotide | SEQ ID NO: 466 |
| Diurnal AMP 65 | Zea mays | Polypeptide | SEQ ID NO: 467 |
| Diurnal AMP 66 | Zea mays | Polynucleotide | SEQ ID NO: 468 |
| Diurnal AMP 66 | Zea mays | Polypeptide | SEQ ID NO: 469 |
| Diurnal AMP 67 | Zea mays | Polynucleotide | SEQ ID NO: 470 |
| Diurnal AMP 67 | Zea mays | Polypeptide | SEQ ID NO: 471 |

### Construction of Nucleic Acids

The isolated nucleic acids of the present disclosure can be made using: (a) standard recombinant methods, (b) synthetic techniques or combinations thereof. In some embodiments, the polynucleotides of the present disclosure will be cloned, amplified or otherwise constructed from a fungus or bacteria.

The nucleic acids may conveniently comprise sequences in addition to a polynucleotide of the present disclosure. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present disclosure. For example, a hexa-histidine marker sequence provides a convenient means to purify the proteins of the present disclosure. The nucleic acid of the present disclosure, excluding the polynucleotide sequence, is optionally a vector, adapter or linker for cloning and/or expression of a polynucleotide of the present disclosure. Additional sequences may be added to such cloning and/or expression sequences to optimize their function in cloning and/or expression, to aid in isolation of the polynucleotide or to improve the introduction of the polynucleotide into a cell. Typically, the length of a nucleic acid of the present disclosure less the length of its polynucleotide of the present disclosure is less than 20 kilobase pairs, often less than 15 kb and frequently less than 10 kb. Use of cloning vectors, expression vectors, adapters and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3′SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSlox and lambda MOSElox. Optional vectors for the present disclosure, include but are not limited to, lambda ZAP II and pGEX. For a description of various nucleic acids see, e.g., Stratagene Cloning Systems, Catalogs 1995, 1996, 1997 (La Jolla, Calif.) and, Amersham Life Sciences, Inc, Catalog '97 (Arlington Heights, Ill.).

### Synthetic Methods for Constructing Nucleic Acids

The isolated nucleic acids of the present disclosure can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68 and the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G> 7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present disclosure provides 5′ and/or 3′ UTR regions for modulation of translation of heterologous coding sequences.

Further, the polypeptide-encoding segments of the polynucleotides of the present disclosure can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present disclosure can be analyzed statistically using commercially available software packages such as “Codon Preference” available from the University of Wisconsin Genetics Computer Group. See, Devereaux, et al., (1984) Nucleic Acids Res. 12:387-395; or MacVector 4.1 (Eastman Kodak Co., New Haven, Conn.). Thus, the present disclosure provides a codon usage frequency characteristic of the coding region of at least one of the polynucleotides of the present disclosure. The number of polynucleotides (3 nucleotides per amino acid) that can be used to determine a codon usage frequency can be any integer from 3 to the number of polynucleotides of the present disclosure as provided herein. Optionally, the polynucleotides will be full-length sequences. An exemplary number of sequences for statistical analysis can be at least 1, 5, 10, 20, 50 or 100.

### Sequence Shuffling

The present disclosure provides methods for sequence shuffling using polynucleotides of the present disclosure, and compositions resulting therefrom. Sequence shuffling is described in PCT Publication Number 96/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present disclosure further provides recombinant expression cassettes comprising a nucleic acid of the present disclosure. A nucleic acid sequence coding for the desired polynucleotide of the present disclosure, for example a cDNA or a genomic sequence encoding a polypeptide long enough to code for an active protein of the present disclosure, can be used to construct a recombinant expression cassette which can be introduced into the desired host cell. A recombinant expression cassette will typically comprise a polynucleotide of the present disclosure operably linked to transcriptional initiation regulatory sequences which will direct the transcription of the polynucleotide in the intended host cell, such as tissues of a transformed plant.

For example, plant expression vectors may include: (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present disclosure in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the 35S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30) and maize H3 histone (Lepetit, et al., (1992) Mol. Gen. Genet. 231:276-85 and Atanassvoa, et al., (1992) Plant Journal 2(3):291-300); ALS promoter, as described in PCT Application Publication Number WO 1996/30530; GOS2 (U.S. Pat. No. 6,504,083) and other transcription initiation regions from various plant genes known to those of skill. For the present disclosure ubiquitin is the preferred promoter for expression in monocot plants.

Alternatively, the plant promoter can direct expression of a polynucleotide of the present disclosure in a specific tissue or may be otherwise under more precise environmental or developmental control. Such promoters are referred to here as “inducible” promoters (Rab17, RAD29). Environmental conditions that may effect transcription by inducible promoters include pathogen attack, anaerobic conditions or the presence of light. Examples of inducible promoters are the Adh1 promoter, which is inducible by hypoxia or cold stress, the Hsp70 promoter, which is inducible by heat stress and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes or alternatively from another plant gene or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50 and An, et al., (1989) Plant Cell 1:115-22) and the CaMV 19S gene (Mogen, et al., (1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119 and hereby incorporated by reference) or signal peptides which target proteins to the plastids such as that of rapeseed enoyl-Acp reductase (Verwaert, et al., (1994) Plant Mol. Biol. 26:189-202) are useful in the disclosure. The barley alpha amylase signal sequence fused to the diurnal polynucleotide is the preferred construct for expression in maize for the present disclosure.

The vector comprising the sequences from a polynucleotide of the present disclosure will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11 and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present disclosure, one may express a protein of the present disclosure in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present disclosure. No attempt to describe in detail the various methods known for the expression of proteins in prokaryotes or eukaryotes will be made.

In brief summary, the expression of isolated nucleic acids encoding a protein of the present disclosure will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present disclosure. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level” or about 1/10 transcripts to about 1/100 transcripts to about 1/1,000 transcripts.

One of skill would recognize that modifications could be made to a protein of the present disclosure without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present disclosure are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present disclosure.

### Expression in Eukaryotes

A variety of eukaryotic expression systems such as yeast, insect cell lines, plant and mammalian cells, are known to those of skill in the art. As explained briefly below, the present disclosure can be expressed in these eukaryotic systems. In some embodiments, transformed/transfected plant cells, as discussed infra, are employed as expression systems for production of the proteins of the instant disclosure.

Synthesis of heterologous proteins in yeast is well known. Sherman, et al., (1982) METHODS IN YEAST GENETICS, Cold Spring Harbor Laboratory is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present disclosure, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present disclosure can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present disclosure are available, for instance, from the American Type Culture Collection Catalogue of Cell Lines and Hybridomas (7^{th} ed., 1992).

Appropriate vectors for expressing proteins of the present disclosure in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenlyation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for diurnal expression placed in the appropriate plant expression vector can be used to transform plant cells. The polypeptide can then be isolated from plant callus or the transformed cells can be used to regenerate transgenic plants. Such transgenic plants can be harvested and the appropriate tissues (seed or leaves, for example) can be subjected to large scale protein extraction and purification techniques.

### Plant Transformation Methods

Numerous methods for introducing foreign genes into plants are known and can be used to insert a diurnal polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods eds. Gamborg and Phillips, Springer-Verlag Berlin Heidelberg N.Y., 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 1991/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209; Longman, N Y (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present disclosure including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Application Number 604 662 A1 discloses a method for transforming monocots using Agrobacterium. EP Patent Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, Theor. Appl. Genet. 69:235-40 (1985); U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a Diurnal Polypeptide Encoded by Diurnal Polynucleotides

Methods are provided to increase the activity and/or level of the diurnal polypeptides encoded by the diurnal polynucleotides of the disclosure. An increase in the level and/or activity of the diurnal polypeptide of the disclosure can be achieved by providing to the plant a diurnal polypeptide. The diurnal polypeptide can be provided by introducing the amino acid sequence encoding the diurnal polypeptide into the plant, introducing into the plant a nucleotide sequence encoding a diurnal polypeptide or alternatively by modifying a genomic locus encoding the diurnal polypeptide of the disclosure.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having cell number regulator activity. It is also recognized that the methods of the disclosure may employ a polynucleotide that is not capable of directing, in the transformed plant, the expression of a protein or an RNA. Thus, the level and/or activity of a diurnal polypeptide may be increased by altering the gene encoding the diurnal polypeptide or its promoter. See, e.g., Kmiec, U.S. Pat. No. 5,565,350; Zarling, et al., PCT/US93/03868. Therefore mutagenized plants that carry mutations in diurnal genes, where the mutations increase expression of the diurnal gene or increase the plant growth and/or organ development activity of the encoded diurnal polypeptide are provided.

Reducing the Activity and/or Level of a Diurnal Polypeptide

Methods are provided to reduce or eliminate the activity of a diurnal polypeptide of the disclosure by transforming a plant cell with an expression cassette that expresses a polynucleotide that inhibits the expression of the diurnal polypeptide. The polynucleotide may inhibit the expression of the diurnal polypeptide directly, by preventing translation of the diurnal messenger RNA, or indirectly, by encoding a polypeptide that inhibits the transcription or translation of a diurnal gene encoding a diurnal polypeptide. Methods for inhibiting or eliminating the expression of a gene in a plant are well known in the art, and any such method may be used in the present disclosure to inhibit the expression of a diurnal polypeptide.

In accordance with the present disclosure, the expression of a diurnal polypeptide is inhibited if the protein level of the diurnal polypeptide is less than 70% of the protein level of the same diurnal polypeptide in a plant that has not been genetically modified or mutagenized to inhibit the expression of that diurnal polypeptide. In particular embodiments of the disclosure, the protein level of the diurnal polypeptide in a modified plant according to the disclosure is less than 60%, less than 50%, less than 40%, less than 30%, less than 20%, less than 10%, less than 5% or less than 2% of the protein level of the same diurnal polypeptide in a plant that is not a mutant or that has not been genetically modified to inhibit the expression of that diurnal polypeptide. The expression level of the diurnal polypeptide may be measured directly, for example, by assaying for the level of diurnal polypeptide expressed in the plant cell or plant, or indirectly, for example, by measuring the plant growth and/or organ development activity of the diurnal polypeptide in the plant cell or plant, or by measuring the biomass in the plant. Methods for performing such assays are described elsewhere herein.

In other embodiments of the disclosure, the activity of the diurnal polypeptides is reduced or eliminated by transforming a plant cell with an expression cassette comprising a polynucleotide encoding a polypeptide that inhibits the activity of a diurnal polypeptide. The plant growth and/or organ development activity of a diurnal polypeptide is inhibited according to the present disclosure if the plant growth and/or organ development activity of the diurnal polypeptide is less than 70% of the plant growth and/or organ development activity of the same diurnal polypeptide in a plant that has not been modified to inhibit the plant growth and/or organ development activity of that diurnal polypeptide. In particular embodiments of the disclosure, the plant growth and/or organ development activity of the diurnal polypeptide in a modified plant according to the disclosure is less than 60%, less than 50%, less than 40%, less than 30%, less than 20%, less than 10% or less than 5% of the plant growth and/or organ development activity of the same diurnal polypeptide in a plant that that has not been modified to inhibit the expression of that diurnal polypeptide. The plant growth and/or organ development activity of a diurnal polypeptide is “eliminated” according to the disclosure when it is not detectable by the assay methods described elsewhere herein. Methods of determining the plant growth and/or organ development activity of a diurnal polypeptide are described elsewhere herein.

In other embodiments, the activity of a diurnal polypeptide may be reduced or eliminated by disrupting the gene encoding the diurnal polypeptide. The disclosure encompasses mutagenized plants that carry mutations in diurnal genes, where the mutations reduce expression of the diurnal gene or inhibit the plant growth and/or organ development activity of the encoded diurnal polypeptide.

Thus, many methods may be used to reduce or eliminate the activity of a diurnal polypeptide. In addition, more than one method may be used to reduce the activity of a single diurnal polypeptide. Non-limiting examples of methods of reducing or eliminating the expression of diurnal polypeptides are given below.

1. Polynucleotide-Based Methods:

In some embodiments of the present disclosure, a plant is transformed with an expression cassette that is capable of expressing a polynucleotide that inhibits the expression of a diurnal polypeptide of the disclosure. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one diurnal polypeptide is an expression cassette capable of producing an RNA molecule that inhibits the transcription and/or translation of at least one diurnal polypeptide of the disclosure. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a diurnal polypeptide are given below.

i. Sense Suppression/Cosuppression

In some embodiments of the disclosure, inhibition of the expression of a diurnal polypeptide may be obtained by sense suppression or cosuppression. For cosuppression, an expression cassette is designed to express an RNA molecule corresponding to all or part of a messenger RNA encoding a diurnal polypeptide in the “sense” orientation. Over expression of the RNA molecule can result in reduced expression of the native gene. Accordingly, multiple plant lines transformed with the cosuppression expression cassette are screened to identify those that show the greatest inhibition of diurnal polypeptide expression.

The polynucleotide used for cosuppression may correspond to all or part of the sequence encoding the diurnal polypeptide, all or part of the 5′ and/or 3′ untranslated region of a diurnal polypeptide transcript or all or part of both the coding sequence and the untranslated regions of a transcript encoding a diurnal polypeptide. In some embodiments where the polynucleotide comprises all or part of the coding region for the diurnal polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657, each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See U.S. Pat. Nos. 5,283,184 and 5,034,323, herein incorporated by reference.

ii. Antisense Suppression

In some embodiments of the disclosure, inhibition of the expression of the diurnal polypeptide may be obtained by antisense suppression. For antisense suppression, the expression cassette is designed to express an RNA molecule complementary to all or part of a messenger RNA encoding the diurnal polypeptide. Over expression of the antisense RNA molecule can result in reduced expression of the native gene. Accordingly, multiple plant lines transformed with the antisense suppression expression cassette are screened to identify those that show the greatest inhibition of diurnal polypeptide expression.

The polynucleotide for use in antisense suppression may correspond to all or part of the complement of the sequence encoding the diurnal polypeptide, all or part of the complement of the 5′ and/or 3′ untranslated region of the diurnal transcript or all or part of the complement of both the coding sequence and the untranslated regions of a transcript encoding the diurnal polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the disclosure, inhibition of the expression of a diurnal polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of diurnal polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the disclosure, inhibition of the expression of one or a diurnal polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., shows 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the diurnal polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the disclosure is catalytic RNA or has ribozyme activity specific for the messenger RNA of the diurnal polypeptide. Thus, the polynucleotide causes the degradation of the endogenous messenger RNA, resulting in reduced expression of the diurnal polypeptide. This method is described, for example, in U.S. Pat. No. 4,987,071, herein incorporated by reference.

vii. Small Interfering RNA or Micro RNA

In some embodiments of the disclosure, inhibition of the expression of a diurnal polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of diurnal expression, the 22-nucleotide sequence is selected from a diurnal transcript sequence and contains 22 nucleotides of said diurnal sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

2. Polypeptide-Based Inhibition of Gene Expression

In one embodiment, the polynucleotide encodes a zinc finger protein that binds to a gene encoding a diurnal polypeptide, resulting in reduced expression of the gene. In particular embodiments, the zinc finger protein binds to a regulatory region of a diurnal gene. In other embodiments, the zinc finger protein binds to a messenger RNA encoding a diurnal polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355, each of which is herein incorporated by reference.

3. Polypeptide-Based Inhibition of Protein Activity

In some embodiments of the disclosure, the polynucleotide encodes an antibody that binds to at least one diurnal polypeptide and reduces the activity of the diurnal polypeptide. In another embodiment, the binding of the antibody results in increased turnover of the antibody-diurnal complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present disclosure, the activity of a diurnal polypeptide is reduced or eliminated by disrupting the gene encoding the diurnal polypeptide. The gene encoding the diurnal polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced cell number regulator activity.

i. Transposon Tagging

In one embodiment of the disclosure, transposon tagging is used to reduce or eliminate the diurnal activity of one or more diurnal polypeptide. Transposon tagging comprises inserting a transposon within an endogenous diurnal gene to reduce or eliminate expression of the diurnal polypeptide. “diurnal gene” is intended to mean the gene that encodes a diurnal polypeptide according to the disclosure.

In this embodiment, the expression of one or more diurnal polypeptide is reduced or eliminated by inserting a transposon within a regulatory region or coding region of the gene encoding the diurnal polypeptide. A transposon that is within an exon, intron, 5′ or 3′ untranslated sequence, a promoter or any other regulatory sequence of a diurnal gene may be used to reduce or eliminate the expression and/or activity of the encoded diurnal polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

ii. Mutant Plants with Reduced Activity

Additional methods for decreasing or eliminating the expression of endogenous genes in plants are also known in the art and can be similarly applied to the instant disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant disclosure. See, McCallum, et al., (2000) Nat. Biotechnol. 18:455-457, herein incorporated by reference.

Mutations that impact gene expression or that interfere with the function of the encoded protein are well known in the art. Insertional mutations in gene exons usually result in null-mutants. Mutations in conserved residues are particularly effective in inhibiting the cell number regulator activity of the encoded protein. Conserved residues of plant diurnal polypeptides suitable for mutagenesis with the goal to eliminate cell number regulator activity have been described. Such mutants can be isolated according to well-known procedures, and mutations in different diurnal loci can be stacked by genetic crossing. See, for example, Gruis, et al., (2002) Plant Cell 14:2863-2882.

In another embodiment of this disclosure, dominant mutants can be used to trigger RNA silencing due to gene inversion and recombination of a duplicated gene locus. See, for example, Kusaba, et al., (2003) Plant Cell 15:1455-1467.

The disclosure encompasses additional methods for reducing or eliminating the activity of one or more diurnal polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 1998/49350, WO 1999/07865, WO 1999/25821 and Beetham, et al., (1999) Proc. Natl. Acad. Sci. USA 96:8774-8778, each of which is herein incorporated by reference.

iii. Modulating Plant Growth and/or Organ Development Activity

In specific methods, the level and/or activity of tissue development in a plant is increased by increasing the level or activity of the diurnal polypeptide in the plant. Methods for increasing the level and/or activity of diurnal polypeptides in a plant are discussed elsewhere herein. Briefly, such methods comprise providing a diurnal polypeptide of the disclosure to a plant and thereby increasing the level and/or activity of the diurnal polypeptide. In other embodiments, a diurnal nucleotide sequence encoding a diurnal polypeptide can be provided by introducing into the plant a polynucleotide comprising a diurnal nucleotide sequence of the disclosure, expressing the diurnal sequence, increasing the activity of the diurnal polypeptide and thereby increasing the number of tissue cells in the plant or plant part. In other embodiments, the diurnal nucleotide construct introduced into the plant is stably incorporated into the genome of the plant.

In other methods, the number of cells and biomass of a plant tissue is increased by increasing the level and/or activity of the diurnal polypeptide in the plant. Such methods are disclosed in detail elsewhere herein. In one such method, a diurnal nucleotide sequence is introduced into the plant and expression of said diurnal nucleotide sequence decreases the activity of the diurnal polypeptide and thereby increasing the plant growth and/or organ development in the plant or plant part. In other embodiments, the diurnal nucleotide construct introduced into the plant is stably incorporated into the genome of the plant.

As discussed above, one of skill will recognize the appropriate promoter to use to modulate the level/activity of a plant growth and/or organ development polynucleotide and polypeptide in the plant. Exemplary promoters for this embodiment have been disclosed elsewhere herein.

Accordingly, the present disclosure further provides plants having a modified plant growth and/or organ development when compared to the plant growth and/or organ development of a control plant tissue. In one embodiment, the plant of the disclosure has an increased level/activity of the diurnal polypeptide of the disclosure and thus has increased plant growth and/or organ development in the plant tissue. In other embodiments, the plant of the disclosure has a reduced or eliminated level of the diurnal polypeptide of the disclosure and thus has decreased plant growth and/or organ development in the plant tissue. In other embodiments, such plants have stably incorporated into their genome a nucleic acid molecule comprising a diurnal nucleotide sequence of the disclosure operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the diurnal polypeptide in the plant. In one method, a diurnal sequence of the disclosure is provided to the plant. In another method, the diurnal nucleotide sequence is provided by introducing into the plant a polynucleotide comprising a diurnal nucleotide sequence of the disclosure, expressing the diurnal sequence and thereby modifying root development. In still other methods, the diurnal nucleotide construct introduced into the plant is stably incorporated into the genome of the plant.

In other methods, root development is modulated by altering the level or activity of the diurnal polypeptide in the plant. An increase in diurnal activity can result in at least one or more of the following alterations to root development, including, but not limited to, larger root meristems, increased in root growth, enhanced radial expansion, an enhanced vasculature system, increased root branching, more adventitious roots and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by increasing the activity and/or level of the diurnal polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by increasing the level and/or activity of the diurnal polypeptide also finds use in promoting in vitro propagation of explants.

Furthermore, higher root biomass production due to an increased level and/or activity of diurnal activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present disclosure further provides plants having modulated root development when compared to the root development of a control plant. In some embodiments, the plant of the disclosure has an increased level/activity of the diurnal polypeptide of the disclosure and has enhanced root growth and/or root biomass. In other embodiments, such plants have stably incorporated into their genome a nucleic acid molecule comprising a diurnal nucleotide sequence of the disclosure operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a diurnal polypeptide of the disclosure. In one embodiment, a diurnal sequence of the disclosure is provided. In other embodiments, the diurnal nucleotide sequence can be provided by introducing into the plant a polynucleotide comprising a diurnal nucleotide sequence of the disclosure, expressing the diurnal sequence and thereby modifying shoot and/or leaf development. In other embodiments, the diurnal nucleotide construct introduced into the plant is stably incorporated into the genome of the plant.

In specific embodiments, shoot or leaf development is modulated by decreasing the level and/or activity of the diurnal polypeptide in the plant. A decrease in diurnal activity can result in at least one or more of the following alterations in shoot and/or leaf development, including, but not limited to, reduced leaf number, reduced leaf surface, reduced vascular, shorter internodes and stunted growth and retarded leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Decreasing diurnal activity and/or level in a plant results in shorter internodes and stunted growth. Thus, the methods of the disclosure find use in producing dwarf plants. In addition, as discussed above, modulation of diurnal activity in the plant modulates both root and shoot growth. Thus, the present disclosure further provides methods for altering the root/shoot ratio. Shoot or leaf development can further be modulated by decreasing the level and/or activity of the diurnal polypeptide in the plant.

Accordingly, the present disclosure further provides plants having modulated shoot and/or leaf development when compared to a control plant. In some embodiments, the plant of the disclosure has an increased level/activity of the diurnal polypeptide of the disclosure, altering the shoot and/or leaf development. Such alterations include, but are not limited to, increased leaf number, increased leaf surface, increased vascularity, longer internodes and increased plant stature, as well as alterations in leaf senescence, as compared to a control plant. In other embodiments, the plant of the disclosure has a decreased level/activity of the diurnal polypeptide of the disclosure.

vi Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the diurnal polypeptide has not been modulated. “Modulating floral development” further includes any alteration in the timing of the development of a plant's reproductive tissue (i.e., a delayed or an accelerated timing of floral development) when compared to a control plant in which the activity or level of the diurnal polypeptide has not been modulated. Macroscopic alterations may include changes in size, shape, number or location of reproductive organs, the developmental time period that these structures form or the ability to maintain or proceed through the flowering process in times of environmental stress. Microscopic alterations may include changes to the types or shapes of cells that make up the reproductive organs.

The method for modulating floral development in a plant comprises modulating diurnal activity in a plant. In one method, a diurnal sequence of the disclosure is provided. A diurnal nucleotide sequence can be provided by introducing into the plant a polynucleotide comprising a diurnal nucleotide sequence of the disclosure, expressing the diurnal sequence and thereby modifying floral development. In other embodiments, the diurnal nucleotide construct introduced into the plant is stably incorporated into the genome of the plant.

In specific methods, floral development is modulated by decreasing the level or activity of the diurnal polypeptide in the plant. A decrease in diurnal activity can result in at least one or more of the following alterations in floral development, including, but not limited to, retarded flowering, reduced number of flowers, partial male sterility and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by increasing the level and/or activity of the diurnal sequence of the disclosure. Such methods can comprise introducing a diurnal nucleotide sequence into the plant and increasing the activity of the diurnal polypeptide. In other methods, the diurnal nucleotide construct introduced into the plant is stably incorporated into the genome of the plant. Increasing expression of the diurnal sequence of the disclosure can modulate floral development during periods of stress. Such methods are described elsewhere herein. Accordingly, the present disclosure further provides plants having modulated floral development when compared to the floral development of a control plant. Compositions include plants having an increased level/activity of the diurnal polypeptide of the disclosure and having an altered floral development. Compositions also include plants having an increased level/activity of the diurnal polypeptide of the disclosure wherein the plant maintains or proceeds through the flowering process in times of stress.

Methods are also provided for the use of the diurnal sequences of the disclosure to increase seed size and/or weight. The method comprises increasing the activity of the diurnal sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for decreasing seed size and/or seed weight in a plant comprises decreasing diurnal activity in the plant. In one embodiment, the diurnal nucleotide sequence can be provided by introducing into the plant a polynucleotide comprising a diurnal nucleotide sequence of the disclosure, expressing the diurnal sequence and thereby decreasing seed weight and/or size. In other embodiments, the diurnal nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development and relates to the successful establishment, after germination, of a well-developed root system and a well-developed photosynthetic apparatus. In addition, an increase in seed size and/or weight can also result in an increase in plant yield when compared to a control.

Accordingly, the present disclosure further provides plants having an increased seed weight and/or seed size when compared to a control plant. In other embodiments, plants having an increased vigor and plant yield are also provided. In some embodiments, the plant of the disclosure has an increased level/activity of the diurnal polypeptide of the disclosure and has an increased seed weight and/or seed size. In other embodiments, such plants have stably incorporated into their genome a nucleic acid molecule comprising a diurnal nucleotide sequence of the disclosure operably linked to a promoter that drives expression in the plant cell.

vii. Method of Use for Diurnal Promoter Polynucleotides

The polynucleotides comprising the diurnal promoters disclosed in the present disclosure, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the diurnal promoter polynucleotides of the disclosure are provided in expression cassettes along with a polynucleotide sequence of interest for expression in the host cell of interest. As discussed in the Examples section of the disclosure, the diurnal promoter sequences of the disclosure are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the disclosure, heterologous sequence expression is controlled by a synthetic hybrid promoter comprising the diurnal promoter sequences of the disclosure, or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic diurnal promoter sequence may comprise duplications of the upstream promoter elements found within the diurnal promoter sequences.

It is recognized that the promoter sequence of the disclosure may be used with its native diurnal coding sequences. A DNA construct comprising the diurnal promoter operably linked with its native diurnal gene may be used to transform any plant of interest to bring about a desired phenotypic change, such as modulating cell number, modulating root, shoot, leaf, floral and embryo development, stress tolerance and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading, and the like.

In certain embodiments the nucleic acid sequences of the present disclosure can be used in combination (“stacked”) with other polynucleotide sequences of interest in order to create plants with a desired phenotype. The combinations generated can include multiple copies of any one or more of the polynucleotides of interest. The polynucleotides of the present disclosure may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene) and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present disclosure with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem. 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996 and WO 98/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502, herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

viii. Identification of Additional Cis-Acting Elements

Additional cis-elements for the diurnal promoters disclosed herein can be identified by a number of standard techniques, including for example, nucleotide deletion analysis, i.e., deleting one or more nucleotides from the 5′ end or internal to a promoter and assaying for regulatory activity, DNA binding protein analysis using DNase I footprinting, methylation interference, electrophoresis mobility-shift assays, in vivo genomic footprinting by ligation-mediated PCR, and other conventional assays or by DNA sequence similarity analysis with other known cis-element motifs by conventional DNA sequence comparison methods and by statistical methods such as hidden Markov model (HMM). cis-elements can be further analyzed by mutational analysis of one or more nucleotides or by other conventional methods.

ix. Chimeric Promoters

Chimeric promoters that combine one or more cis-elements are known (see, Venter, et al., (2008), Trends in Plant Science, 12(3):118-124). Chimeric promoters that contain cis-elements from the promoters disclosed herein along with their flanking sequences can be engineered into other promoters that are for example, tissue specific. For example, a chimeric promoter may be generated by fusing a first promoter fragment containing the activator (diurnal) cis-element from one promoter to a second promoter fragment containing the activator (tissue-specific) cis-element from another promoter; the resultant chimeric promoter may increase gene expression of the linked transcribable polynucleotide molecule in both diurnal and tissue specific manner. Regulatory elements disclosed herein are used to engineer chimeric promoters, for example, by placing such an element upstream of a minimal promoter.

This disclosure can be better understood by reference to the following non-limiting examples. It will be appreciated by those skilled in the art that other embodiments of the disclosure may be practiced without departing from the spirit and the scope of the disclosure as herein disclosed and claimed.

### EXAMPLES

### Example 1

### Diurnal Studies in Maize

Maize plants (B73 genotype) were grown under field conditions and sampled at the reproductive V14-15 stage. Light conditions at sampling were approximately 14.75 hours of sunlight according to records of US Naval Observatory (Materials and Methods). Starting at sunrise on day 1, the top leaves and immature ears were sampled at 4 hour time intervals over three consecutive days. RNA profiling was performed on custom Agilent Maize arrays designed to interrogate global gene expression patterns across circa 105K probes. Samples for the Illumine Digital Gene Expression (DGE) platform were collected with 3 replicate pools of 3 plants every 4 hours over a 1 day period. The three samples were then split into three groups for analysis.

The GeneTS methodology was applied to the data to determine periodicity (Wichert, et al., (2004) Bioinformatics 20:5-20). This method first creates a periodogram for Fourier frequencies. Significant Fourier frequencies are then assessed for significance via Fisher's g-statistic. Given the experimental design, this method shows greater power in the detection of circadian rhythmicity than other commonly used methods (Hughes, et al., (2007) Cold Spring Harb Symp Quant Biol 72:381-386; Hughes, et al., (2009) PLoS Genet 5:e1000442). The significance values from Fisher's G-Test were then corrected for multiple measures comparisons via conversion to q-values to assess False Discovery Rates (Storey and Tibshirani, (2003) Proc Natl Acad Sci USA 100:9440-9445). Diurnally regulated transcripts were determined as those having significant expression at least once per day and also that were significant at a FDR rate of 10%.

### Leaf Diurnal MicroArray Analysis

Diurnal rhythms of gene expression were readily detectable within the photosynthetic leaf tissue. Of the 44,187 probes with detectable expression, 10,037 or 22.7% were identified as cycling by the GeneTS algorithm. This proportion of cycling transcripts is in line with the proportion reported for Arabidopsis (Hazen, et al., (2009) Genome Biol 10:R17). Significantly cycling transcripts have a median period of 24.1 hours, as would be expected for natural conditions. Amplitudes of cycling transcripts are robust, with a median peak/trough ratio ˜5-fold, with many showing peak/trough ratios of higher than 20-fold. The peak expression for these cycling transcripts exhibits a broad distribution, peaking at all phases of the day.

### Ear Diurnal MicroArray Analysis

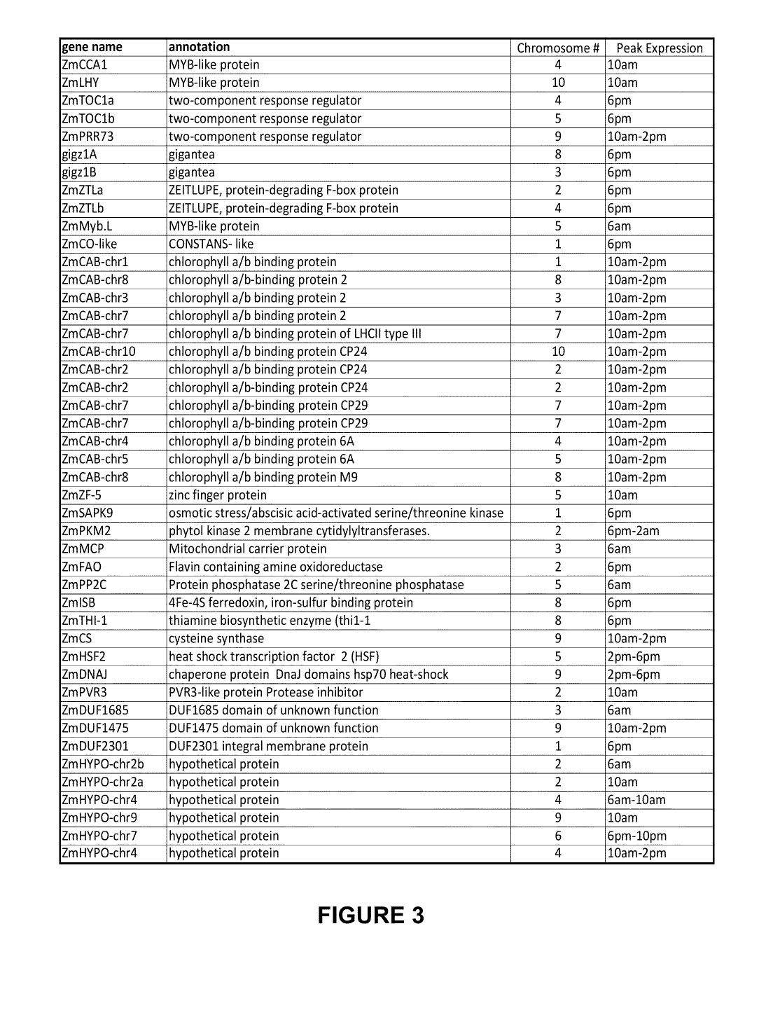

In contrast to the leaf results, very few transcripts within the developing ear exhibited diurnal rhythms. Only 149 of the 38,445 expressed transcript probes (1.7%) were positively identified as cycling. Despite the low numbers of cycling transcripts, there is early-evening enrichment, with roughly half of the cycling transcripts peaking in this phase. Of the 149 transcripts, 100 (67.1%) were also diurnally cycling in the leaf tissue. Among those that cycled in both leaf and ear tissues, the amplitudes of the rhythms is severely attenuated in the developing ear. This list was reduced to 45 putative ear cycling genes after consolidation of redundant probes and more thorough gene annotation (FIG. 3). Many of these genes appeared to be maize homologues of well-described Arabidopsis oscillators CCA1/LHY, TOC1, PRR7/3, GI, ZTL (ZEITLUPE, known as Adagio-like protein 3 in rice). The maize ear tissue core oscillator thus appears to be intact, but is apparently decoupled from the majority of its transcriptional output systems.

A few output genes are nonetheless found in the set of genes that cycle in ears. The list of robust cycling transcripts include up to 13 maize light-harvesting CAB transcripts (chlorophyll a-b binding protein), which is a subset of the greater maize CAB gene family. The CONSTANS-like (ZmCO-like) gene, mapped to chromosome 1, cycles in ears and leaves with a peak of expression at early evening (6 PM). However it is a different CO homologue that have been previously identified as conz1 on chromosome 9 (Miller, et al., (2008) Planta 227:1377-1388).

Robust cycling was detected for the MYB-like transcription factor (ZmMyb.L) which peaked at dawn (6 AM). This gene is a homologue of REVEILLE1, a Myb-transcription factor integrating the circadian clock and auxin pathway in Arabidopsis (Rawat, et al., (2009)). Two ear-specific genes have intriguing putative functions, a zinc finger protein (ZmZF-5) peaking at 10 AM and an osmotic stress/abscisic acid-activated serine/threonine-protein kinase (ZmSAPK9) peaking at 6 PM. Among other cycling genes there are three encoding transporters, two heat shock proteins, several enzymes and hypothetical proteins.

### Digital Gene Expression Analysis

Independent samples were taken specifically for the Illumina DGE expression platform (Illumina, Inc., 9885 Towne Centre Drive, San Diego, Calif. 92121 USA), were also analyzed for rhythmicity. This represents the first NexGen-style deep sequencing effort for determining rhythmic diurnal expression patterns. Three replicates from each of six time points (ZT0, ZT4, ZT8, ZT12, ZT16 and ZT20) were sequenced off anchor points for two restriction enzyme cut sites, DPNII and NLAIII. Each multiplexed sample was run in separate flow cell lanes. Output sequences were assessed for quality and aligned against the Dana Farber Gene Index Maize 19.0 (found on world wide web at compbio.dfci.harvard.edu/tgi/). A total of 4.7×10⁹ base pairs passed all quality control and alignment measures from the sequencing runs, which is approximately 1.3×10⁸ bp per lane. Over 1.89×10⁸ tags were advanced for gene expression analysis of rhythmic behavior, or roughly 5.25 million tags per sample. The three replicates were artificially split into three consecutive days. The data was then assessed for periodicity in the same manner as the microarray data. This data is chiefly used here as independent confirmation for those cycling transcripts identified through the more statistically robust microarray strategy, and therefore it is not here used as a stand-alone discovery experiment.

The results show broad concordance with the Agilent analysis. In the leaf tissue, 2559 transcripts were identified as cycling in the leaf tissue. All of the core components identified as cycling by Agilent were also determined to be cycling under Illumina. There were 1378 transcripts that were identified as cycling by both technologies. As these transcripts were independently found by each distinct profiling platform, these transcripts serve as the most confident base set for cycling transcripts in photosynthetic (leaf) tissues of maize.

The developing ear Illumina profiling showed over twice as many cycling transcripts than Agilent, with 362 showing significant rhythms. Yet, while the number of cycling genes in developing ear increased, it remained small in comparison to the leaf photosynthetic tissue. Though the concordance between these distinct technologies was lower, 48 transcripts were still identified from ears as cycling in both platforms. Of these 48 transcripts that did cycle, 23 were identified by both the Agilent and Illumina technologies and in both leaf and ear tissues. Of the remaining 25, 24 were identified as cycling in three out of the four possible tests (Leaf Agilent, Leaf Illumina, Ear Agilent and Ear Illumina). These independent results confirm that the core oscillator is functioning in ear tissue.

### Diurnal Expression Analysis

The diurnal transcriptional profiles of maize are robust and similar to that of the model plant Arabidopsis in independent biological tissues and technical platforms. Results from the light receiving photosynthetic leaf tissue identified diurnal rhythms for as high as 22.7% (10K/44K probes) of the expressed transcripts using the Agilent technology. Using two independent transcriptome-wide analysis platforms, Agilent Microarrays and Illumina Tag Sequencing, compensates for the biases inherent to either technology and reveals a minimal core high confidence set of 1400 transcripts that are diurnally regulated.

In the non-photosynthetic developing ear, diurnal rhythms were not a significant contributor to the transcriptional program. Just 45 genes were identified as cycling either in ears only or in both ears and leaves. Among them 13 CAB (chlorophyll A/B transcripts) were found, now well-established markers of diurnal expression patterns in plants (Millar and Kay, (1991) Plant Cell 3:541-550). However, their amplitudes were severely attenuated in ears as compared to leaves. Eleven orthologs of the core oscillator system appear in this cross-tissue leaf-ear set. Therefore it appears that the core oscillator is active in ears. The core oscillator of plants has been described as an interlocking three or four loop process (Harmer, (2009) supra; Ueda, (2006) Mol Syst Biol 2:60). The results indicate that the central feedback loop, consisting of ZmCCA1/ZmLHY and ZmTOC1a,b is conserved in maize. This loop shows extreme amplitude waves in leaf tissue and likely serves as the main driver for transcriptional output. In the ear tissue, the amplitude of these waves are attenuated, reduced 83% and 94% respectively, mainly by a reduction in peak transcriptional levels. The reduced height of the ear tissue wave pattern strongly points to persistent diurnal cycling but at decreased amplitude. It does not appear to be a de-synchronization of the diurnal pattern that might spread offsets in cycling patterns so as to mute or obscure the peak-trough wave pattern. If the ZmCCA1/ZmTOC1 loop does serve as the central zeitgeber, with its attenuated wave pattern, its relative contribution to signaling diurnal output genes should be severely reduced. The two exterior loops, containing such genes as ZmPRR73/ZmPRR37, gigz1/gigz2 and ZmZTLa/ZmZTLb also show significant reductions in wave amplitude.

One explanation for the decoupling of the core machinery from the output pathways in ears could be attributed to the low light intensity penetrating developing ears through the husk leaves (bracts) that are wrapped around ears. Transcriptional reinforcement of the diurnal expression pattern may occur via light sensing proteins such as the phytochromes and cryptochromes and therefore this reinforcement would be reduced accordingly in ears experiencing a relative absence of light. As shown in Arabidopsis, the core oscillator clock genes, such as CCA1 and LHY, are activated by light and mediate activation of the output CAB genes (Wang, et al., (1997) supra). The low amplitude of the core oscillators may therefore not generate enough protein to trigger transcription of the output pathways or do so feebly. A few output genes whose promoters might be sensitive to lower levels of the core oscillator products are activated but the overall transcriptional outputs has been effectively decoupled.

In ears there are few cycling genes that may be proximal translational nodes connecting the core oscillator to the output pathways. One of them is ZmMyb.L which has a peak of expression at 6 AM in leaves and ears. The ZmMYB.L protein shows a high degree of identity to the MYB domain of the morning phase genes CCA/LHY of both Arabidopsis and maize, extending even to including the distinctive SHAQKYFF protein motif. ZmMyb.L might have the orthologous function of Arabidopsis REVEILLE1, that integrates the circadian clock with the auxin pathway (Rawat, (2009) supra).

Microarray analysis of Arabidopsis root and shoot tissue grown has shown that a simplified version of the core oscillator does cycle in root non-photosynthetic tissue (James, et al., (2008) Science 322:1832-1835). According to that microarray expression study, 6518 transcripts are identified as cycling in shoot tissue compared with 335 in the root tissue. Those results largely agree with the hereby disclosed findings; that is, in largely non-photosynthetic tissues, whether root or ear, many components of the core oscillator function, but their transcriptional output is largely attenuated.

### Diurnal Physiological Functions

Diurnal gene expression rhythms were studied in order to better understand the scope of diurnally regulated biology at the molecular level that could lead to opportunities to improve crop plant performance. (FIG. 5) These results reveal many aspects of the maize diurnal mechanism, from core clock genes, signaling and downstream effecter genes. The diurnal swing in maize leaf gene expression is pervasive, with thousands of genes and their attendant functions cycling in a diurnal tide. The apparent succession of physiological roles across the span of the day is intriguing and suggests specifically staged control of expression, but this may also be a natural progression of physiological events unfolding in response to both proximal and distal events in the diurnal rhythm. It is acknowledged that finer timepoint resolution will yield both more diurnally regulated transcripts, and also better delineate the succession of functional focusing across the day. This genome-wide diurnal profiling survey, the first for maize, coupled with assignments to over 1700 functional terms, has uncovered a durable outline of the succession of functional events in the day. It is clear that diurnal rhythms are complex and deeply woven into the biology of the cell and presumably it is adaptive to have coincident or coordinated expression of cellular machinery.

The presence of the bimodal functional enrichment pattern in the morning and afternoon/evening is intriguing and almost certainly reflects a fundamental activity in the plants daily regimen. More genes are peaking at the 10 AM and 6 PM timepoints and this will by itself cause more functional categories to which these genes belong to also peak at those times, resulting in this bimodal functional pattern. Although individual diurnally regulated genes are peaking at just one time during the day, the fact that the functional categories are bimodal, means that different genes under those functional umbrellas are peaking at different times. A possible connection to the recently described ‘solar clock’ that is calibrated to mid-day can now also be considered (Yeang, (2009) Bioessays 31:1211-1218). These morning and evening peaks could signify communication occurring between diurnally regulated genes and solar clock-regulated genes.

The diurnal patterns are strong in leaves, but feeble in developing ears. Developing ears are also the main sink for the photosynthetic source organs experiencing the throws of diurnal swings. Even if immature ears do not themselves have a marked internal diurnal drive, received from source organs might be expected to occur, as via waves of mobile signals and fixed carbon, to stir diurnal transcriptional action of some genes from outside. Yet, this is apparently not observed. Considering the times at which the few ear diurnally regulated genes peak during the day the functional enrichment suggests signal transduction and transcription in the morning, photosynthesis in the afternoon and core oscillator and transcriptional regulation in the evening.

Components of the core clock mechanism and proximal signaling mechanism emanating from it, could be modified in such manner as to positively affect crop performance, as by for example shifting or extending the relationship between sources and sinks such as leaves and ears. Wholesale genetic complementation of diurnal patterns from different germplasm sources has been shown augment the combined diurnal patterns and apparent fitness (Ni, (2009) supra).

### Example 2

### Genomic Structures of ZmCCA1 and ZmLHY

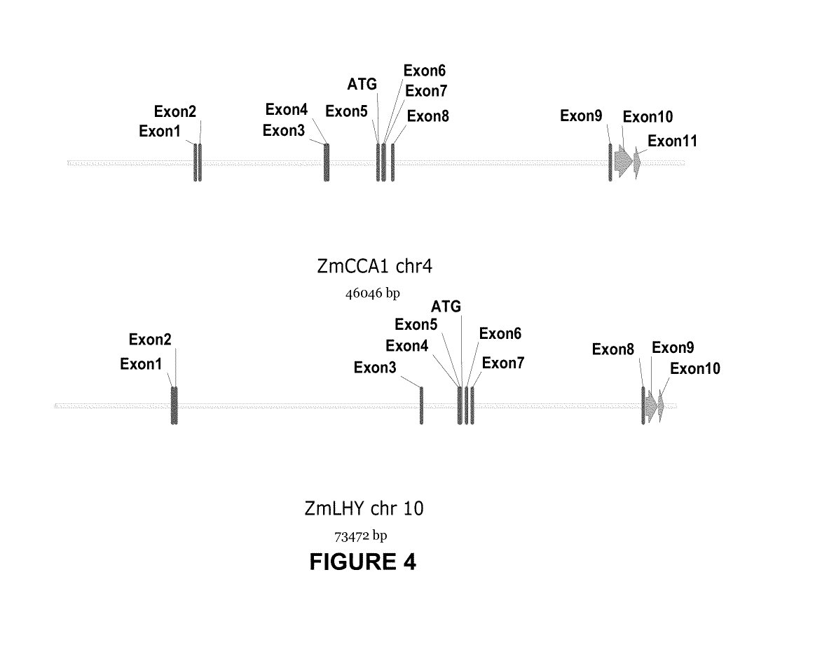

In the course of working out the maize gene models for ZmCCA1 and ZmLHY it was revealed that the genes are encoded by genic regions of circa 45 kb and 78 kb respectively (FIG. 4). Maize genes of this size are extremely rare, where the average gene size is closer to 4 kb (Bruggmann, et al., (2006) Genome Res 16:1241-1251). The exon-intron model of ZmCCA1 and ZmLHY genes was deduced from alignment of their cDNA and genomic sequences obtained from BAC sequencing. The ZmCCA1 gene is composed of 11 exons separated by 10 introns of various lengths. The longest are intron #2 (˜9 kb) and intron #6 (˜15.6 kb) which are rarely seen in maize genome. The translation start codon ATG is located in exon #5. This means that the untranslated 5′ UTR is divided into 5 small exons ranging in the sizes of 40-200 bp. The ZmLHY gene is composed of 10 exons separated by 9 introns of various lengths. (It is likely that one of the small exon is missing in available ESTs). The intron 2 is ˜30.0 kb and intron 6 is ˜20.1 kb that are likely the largest introns in the maize genome. It is known that regulatory sequences controlling gene expression are often located in introns. The unusually long introns may play a role in ZmCCA1 and ZmLHY regulation. Both ZmCCA1 and ZmLHY genes are extremely long. Exceptionally long genes could slow transcription, thereby be a form of genomic regulation of gene expression. Similar to ZmCCA1 the translation start codon ATG is located in the exon 5. The complex exonic structures of the 5′UTRs suggest that maturation of pre-mRNA may be the other level of regulation of these genes.

### DNA Sequencing

The BAC clones were sequenced using the double-stranded random shotgun approach (Bodenteich, et al., Shotgun cloning or the strategy of choice to generate template for high-throughput dideoxynucleotide sequencing, in: M. D. Adams, C. Fields, J. C. Venter (Eds.), Automated DNA Sequencing and Analysis, Academic Press, San Diego, 1994, pp. 42-50). Briefly, after the BAC clones were isolated via a double-acetate cleared lysate protocol, they were sheared by nebulization and the resulting fragments were end-repaired and subcloned into pBluescript II SK(+). After transformation into DH-10B electro-competent Escherichia coli cells (Invitrogen) via electroporation, the colonies were picked with an automatic Q-Bot colony picker (Genetix) and stored at −80° C. in freezing media containing 6% glycerol and 100 m/ml Ampicillin. Plasmids then were isolated, using the Templiphi DNA sequencing template amplification kit method (GE Healthcare). Briefly, the Templiphi method uses bacteriophage φ29 DNA polymerase to amplify circular single-stranded or double-stranded DNA by isothermal rolling circle amplification (Reagin, et al., (2003) J. Biomol. Techniques 14:143-148). The amplified products then were denatured at 95° C. for 10 min and end-sequenced in 384-well plates, using vector-primed M13 oligonucleotides and the ABI BigDye version 3.1 Prism sequencing kit. After ethanol-based cleanup, cycle sequencing reaction products were resolved and detected on Perkin-Elmer ABI 3730xl automated sequencers, and individual sequences were assembled with the public domain Phred/Phrap/Consed package (on the world wide web at:phrap.org/phredphrapconsed.html). Contig order was viewed and confirmed with Exgap (A. Hua, University of Oklahoma, personal communication). Exgap is a local graphic tool that uses pair read information to order contigs generated by Phred, Phrap and Consed, and confirm the accuracy of the Phrap-based assembly. Subsequently, a majority of the sequencing gaps between contigs of interest were closed by sequencing plasmid DNA templates previously amplified with the Templiphi amplification kit method, in the presence of custom-designed sequencing primers and by inserting the resulting custom sequences to the original Phrap-based assemblies. Sequencing overlaps with public BAC DNA sequences (namely, ZMMBBc0099K11 (GenBank AC211312.1) and ZMMBBc0076L18 (GenBank AC213378.3) from the National Center for Biotechnology Information's nucleotide database) also were used to confirm remaining gap sequences between contigs of interest.

### Example 3

### Diurnally Regulated Promoters

Diurnal (day/light) cycles in light and temperature are environmental factors that all living organisms are adapted to. Virtually all aspects of plant physiology such as growth, development, photosynthesis and photo-assimilate partitioning, respiration, stress response, hormone response, nitrogen assimilation are diurnally regulated.

The time-of-the day promoters provide the tools for manipulating the specific physiological or metabolic process in a controlled manner according to the natural diurnal pattern. For example, the artificial down regulation of the morning clock genes CCA1 and LHY during the day will lead to the up-regulation of genes involved in photosynthesis and carbohydrate metabolism boosting the growth vigor and yield. To achieve down regulation the CCA1 and LHY promoters may drive their own RNAi expression cassettes.

The genome wide diurnal RNA profiling provides candidates for promoters for every phase of the day with high-inducibility and low background. Depending on what is needed specific time-of-day examples that are pulsate (i.e., transcribed only briefly once per day), broad peaked (e.g., transcribed 12 h on, 12 h off) or anywhere in between.

Genes involved in a variety of agronomic traits such as, for example, freezing tolerance, chilling or cold tolerance, drought tolerance, yield increase through improved metabolism are suitable for modulation by the diurnal regulatory elements disclosed herein. Optionally, these diurnal elements are used in combination with tissue specific promoters to optimize desired expression pattern of the genes of interest. For example, in an embodiment, genes that improve drought tolerance are expressed under the control of a diurnal regulatory element that exhibits a peak expression pattern around noon or late afternoon and in combination with a root-specific promoter element. Similarly, genes that improve tolerance to chilling and freezing are expressed under the control of a diurnal regulatory element that exhibits a peak expression pattern at dawn or night and in combination with a leaf-specific promoter element. In addition, genes that are involved in carbohydrate metabolism and source/sink relationships during photosynthesis are expressed under the control of diurnal promoter elements disclosed herein in combination with one or more tissue specific promoter elements. A variety of genes are known to be involved in abiotic stress tolerance and nitrogen use efficiency (see, e.g., US Patent Application Publication Numbers US 2010/0223695; US 2010/0313304; US 2010/0269218). As shown in FIG. 5, genes belonging to various functional categories exhibit different diurnal expression pattern. For example, GO:0009651 response to salt stress peaks during mid-morning whereas GO:0008643 carbohydrate transport peaks at night.

Genes that are co-regulated from related pathways with those that are diurnally regulated are also within the scope of this disclosure. Expression of those related pathway members are manipulated to be better regulated through the use of one or more diurnal regulatory elements disclosed herein.

### Promoter Motif Analysis Process

It has been shown in the literature that the combination of just a few motifs, through constructive and destructive interference, can produce waveforms that peak under any phase shift. (such as CBE: Wang, et al., (1997) Plant Cell 9:491-507 and EE: Alabadí, et al., (2001) Science 293:880-883. However, the extent of both the number of these controlling elements and their conservation across plant species has not been adequately addressed. Promoters of the 144 maize genes were grouped by Zeitgeber time, the timing of their peak expression, Where ZT0=6 am, ZT4=10 am, ZT8=2 pm, ZT12=6 pm, ZT16=10 pm and ZT20=2 am. Each group of promoters was analyzed for the existence of motifs identified in the distant species Arabidopsis Thaliana. The motifs were “CBE”, “EE”, “O-G-box”, “Morning Element”, “SORLIP1”, “Refined Morning Consesnus”, “Evening GATA”, “Telo Box”, “Starch Box” and “Protein Box”. These motifs were identified via literature search, and include motifs that have been identified for morning, evening and night expression. Promoters were scanned for exact matches of the motifs in both forward and reverse orientations within 2000 bp of the TSS.

TABLE 2

| ELEMENT | SEQUENCE | SEQ ID |
|---|---|---|
| CBE | AAAAATCT | SEQ ID NO: 472 |
| CBE′ | AGATTTTT | SEQ ID NO: 473 |
| EE | AAATATCT | SEQ ID NO: 474 |
| EE′ | AGATATTT | SEQ ID NO: 475 |
| O G-Box | GCCACGTG | SEQ ID NO: 476 |
| O B-Box′ | CACGTGGC | SEQ ID NO: 477 |
| Morning Element | AACCAC | SEQ ID NO: 478 |
| Morning Element′ | GTGGTT | SEQ ID NO: 479 |
| SORLIP1 | GCCAC | SEQ ID NO: 480 |
| SORLIP1′ | GTGGC | SEQ ID NO: 481 |
| Refined Morning Element | CCACAC | SEQ ID NO: 482 |
| Refined Morning Element′ | GTGTGG | SEQ ID NO: 483 |
| Evening GATA | GGATAAG | SEQ ID NO: 484 |
| Evening GATA′ | CTTATCC | SEQ ID NO: 485 |
| TeloBox | AAACCCT | SEQ ID NO: 486 |
| TeloBox′ | AGGGTTT | SEQ ID NO: 487 |
| Starch Box | AAAGCCC | SEQ ID NO: 488 |
| Starch Box′ | GGGCTTT | SEQ ID NO: 489 |
| Protein Box | ATGGGCC | SEQ ID NO: 490 |
| Protein Box′ | GGCCCAT | SEQ ID NO: 491 |

Circadian motifs were culled from an extensive literature search, including: CBE: Carré and Kay, (1995) Plant Cell 7 2039-2051. EE: Harmer and Kay, (2005) Plant Cell 17 1926-1940. G-BOX, TELO, STARCH, PROTEIN and GATA: Michael, et al., (2008). PLoS Genet. 4e14. SORLIP and Refined Morning Consensus: Hudson and Quail, (2003) Plant Physiol. 133 1605-1616. Morning Element: Harmer and Kay, (2005) Plant Cell 17 1926-1940.

Hidden Markov Models (HMMs) were built for the EE and CBE motifs from several genes containing the motifs that cycled both significantly and in the same appropriate phase as their Arabidopsis ortholog. These HMMs showed no preference for any surrounding bases, hence the exact core motifs were used for further analysis. Exact matches to both the motif and reverse complement were pulled from sequences where present. Both the number of genes and the sum total of motifs found were compared against a random probability and against the rest of the set to search for enrichment.

### Motif Analysis Results

The “CBE motif”, an 8 bp motif also known as the CCA1 Binding Element, should appear at random13 times in a set the size of the current analysis; the exact CBE motif was found 40 times in the 144 promoters. The CBE was enriched in genes found during daylight hours, which follows the expression pattern of the maize ortholog of Arabidopsis thaliana CCA1 (included in this disclosure).

The “EE motif”, an 8 bp motif also known as the Evening Element, should appear at random13 times in a set the size of the current analysis; the exact EE motif was found 34 times in the 144 promoters. Furthermore, the prevalence of the motif was concentrated in those promoters that corresponded to evening and night peaking genes, with >40% of the motifs lying in promoters of the ZT12 group and >70% of the instances lying between 6 pm-2 am. Among those genes with peak expression at ZT12, 12/23 genes contained at least one EE.

The “O-G-Box” has been identified as morning driven motif and the data here show that 50% of all O-G-Box motifs found were for the first time point after the onset of light, ZT4. Other morning elements, “Morning Element”, “SORLIP1” and “Refined Morning Element”, all showed similar patterns, with peak enrichment in those time points immediately after the onset of light (28%, 33% and 31% respectively), consistent with the theory that these promoters are light driven. Also consistent with this is the fact that given the long day period in that plants were grown in to generate the initial data, the presence of these promoters in selected against in the two true-dark time points, ZT16 and ZT20.

The “Evening GATA”, “Telo Box”, “Starch Box” and “Protein Box” motif have all been identified as evening to late night motifs. Here, there is an under-enrichment of these motifs in those timepoints defined as midday, when light is the brightest. The relatively broad spectrum of these elements across all evening and night time points is consistent with the theory of multiple motifs combining to produce different phases of peak expression.

It is important to note that many of the 144 promoters identified carried more than one motif, the median number of motifs found per promoters was 4, and the maximum number of motifs found was 12. Twelve of the promoters contain none of the motifs at all, spanning every time point. In the ZT12 peaking set, which includes the highly prevalent EE motif, 11/23 genes contained no canonical EE and as stated above, several contained no known motifs at all, indicating that other factors and motifs are at play causing the high amplitude observed waveforms, which nonetheless may be contained within the promoter sequences disclosed herein.

### Promoter Expression Analysis

### Seedling Prep

GS3 seeds were sterilized and prepared for germination by washing with 70% ethanol for five minutes, followed a 15 minute wash in a solution of 50% bleach with two drops of Tween® 20. Then three washes in sterile water for 5, 15 and 5 minutes. The seeds were then washed in 30% Hydrogen Peroxide for 5 minutes, then washed 3 times with sterile water. The seeds were then allowed to soak in sterile water for 5 hours.

Sterile germination paper was moistened with 15 ml of sterile water and placed in sterile Q-trays. Sixteen seeds per tray were placed at regular intervals and covered with another sterile germination paper and dampened with 9 ml of sterile water. The Q-tray was sealed with Austraseal tape, and placed in a growth chamber with light at 22° C., and allowed to grow for 3 days.

The pericarp material covering the developing seedling was removed and the germinated seedlings were placed, 2 per plate, on media containing 4.3% MS Basal Salts, 0.1% Myo-inositol, 0.5% MS Vitamin stock and 40% sucrose, at pH 5.6.

### Leaf Prep and Bombardment

One inch wide cross sections were isolated from the youngest leaf (partially emerged) of a 2½ to 3 week old GS3 seedling and placed on media for bombardment containing 4.3% MS Basal Salts, 0.1% Myo-inositol, 0.5% MS Vitamin stock and 40% sucrose, at pH 5.6.

### Embryo Prep

Immature maize embryos from greenhouse donor plants are bombarded with a plasmid containing the GUS gene operably linked to a test promoter. Transformation is performed as follows.

Maize GS3 ears are harvested 8-14 days after pollination and surface sterilized in 30% Chlorox® bleach plus 0.5% Micro detergent for 20 minutes and rinsed two times with sterile water. The immature embryos are excised and placed embryo axis side down (scutellum side up), 25 embryos per plate. These are cultured on 560L medium 4 days prior to bombardment in the dark. Medium 560L is an N6-based medium containing Eriksson's vitamins, thiamine, sucrose, 2,4-D and silver nitrate. The day of bombardment, the embryos are transferred to 560Y medium for 4 hours and are arranged within the 2.5-cm target zone. Medium 560Y is a high osmoticum medium (560L with high sucrose concentration). Following bombardment, the embryos are kept on 560Y medium, an N6 based medium, for 1 day, then stained for GUS expression.

### Bombardment

DNA/gold particle mixtures were prepared for bombardment in the following method: 60 mg of 0.6-1.0 micron gold particles were pre-washed with ethanol, rinsed with sterile distilled H₂O and resuspended in a total of 1 mL of sterile H₂O. DNA was precipitated onto the surface of the gold particles by sonicating 25 μL of pre-washed 0.6 μM gold particles and adding to 20 μL of test plasmid at 100 ng/μL. This mixture was sonicated once again and 2.5 μL of TFX was added. That solution was placed on a vortex shaker for 10 minutes at a low setting. The solution was then centrifuged for 1 min at 10K RPM, and the liquid removed from the tube. 60 μL of ethanol was added, then the solution was sonicated once again. 10 μL of the DNA/gold mixture was then placed onto each macrocarrier and allowed to dry before bombardment.

Seedlings were bombarded using the PDS-1000/He gun at 1100 psi for leaf and seedling tissue and 450 psi for embryos, under 27-28 inches of Hg vacuum. The distance between macrocarrier and stopping screen was between 6 and 8 cm. Plates were incubated in sealed containers for 18-24 h at 27-28° C. following bombardment. Two plates from each construct were incubated in the dark, while two plates were incubated in the light.

The bombarded tissues were assayed for transient GUS expression by immersing the seedlings in GUS assay buffer containing 100 mM NaH₂PO₄—H₂O (pH 7.0), 10 mM EDTA, 0.5 mM K₄Fe(CN)₆-3H₂O, 0.1% Triton X-100 and 2 mM 5-bromo-4-chloro-3-indoyl glucuronide. The tissues were incubated in the dark for 24 h at 37° C. Replacing the GUS staining solution with 70% ethanol stopped the assay. GUS expression/staining was visualized under a microscope.

### BMS Transformation

BMS (Black Mexican Sweet) cells were grown in 250 ml flasks containing 40 ml of #237 media (4.3% MS Basal Salts, 0.1% Myo-inositol, 0.5% MS Vitamin stock, 0.002% 2,4-D and 40% sucrose, at pH 5.6) in the dark at 28° C. and shaking at ˜150 RPM for 3 days. At that time, 25 ml of #237 liquid media was added and the culture was allowed to continue to grow for another 3 days, at which time the agro transformation could take place. One day prior to that, agrobacterium cultures containing a plasmid containing the GUS gene operably linked to a test promoter was place in a 10 ml culture containing the appropriate antibiotic and allowed to grow at 28° C. overnight.

Each 250 mL flask was placed in the laminar flow hood for 10 minutes to allow the cells to settle. 20 ml of supernatant was removed. The remaining mixture was moved to a 50 ml tube and centrifuged at 3200 RPM for 5 min. The supernatant was removed and replaced with 40 ml of 561Q liquid media. 561Q is a 4% N6-based medium containing Eriksson's vitamins (1×), 0.005% Thiamine, 68.5% sucrose, 0.0015% 2,4-D, 0.69% L-Proline and 36% glucose, at pH 5.2. The cells were again centrifuged at 3200 RPM for 5 min. The cells were resuspended to a final volume of 15 ml in 561Q and split into 7.5 ml aliquots in 125 ml flasks.

The agro culture was then centrifuged at 3200 RPM for 5 minutes, the supernatant poured off, and the pellet resuspended in 2 ML of 561Q+Acetosyringine (AS). The Acetosyringine solution was prepared by making a 100 mM solution in DMSO. This solution was added to 561Q at 1 uL A.S./1 mL #561Q. The absorbance at OD550 was measured to determine the concentration of cells to use for transformation. At an OD550 of 0.75, 1 ml of the agro solution was added to 5 ml of 561Q+AS, and that was co-cultured with the 7.5 mls of BMS cells for 3 hours in the dark at 28° C. while shaking at 150 RPM.

After the 3 hour incubation, more 561Q media was added to the 13.5 ml of culture to bring the volume to ˜48 ml in a 50 ml tube. 12 ml of culture was applied to a sterile filter disk, then placed on a plate of 562 U media in the dark at 28° C. for 4 days. 562 U is a 4% N6-based medium containing Eriksson's vitamins (1×), 0.005% Thiamine, 30% sucrose, 0.002% 2,4-D and 0.69% L-Proline, at pH 5.8. The filters were then moved to 563N plates and placed in the dark at 28° C. for an additional 2 days. 563N is a 4% N6-based medium containing Eriksson's vitamins (1×), 0.005% Thiamine, 30% sucrose, 0.0015% 2,4-D, 0.69% L-Proline and 0.5% MES Buffer at pH 5.8.

Four plates were created for each test construct. Two BMS plates from each were pulled from the dark and stained for GUS, while two others were placed in the light for 5 hours before staining for GUS. The BMS cells were scraped from the filter into a new tube and were assayed for transient GUS expression by immersing the cells in GUS assay buffer containing 100 mM NaH₂PO₄—H₂O (pH 7.0), 10 mM EDTA, 0.5 mM K₄Fe(CN)₆-3H₂O, 0.1% Triton X-100 and 2 mM 5-bromo-4-chloro-3-indoyl glucuronide. The tissues were incubated in the dark for 24 h at 37° C. Replacing the GUS staining solution with 70% ethanol stopped the assay. GUS expression/staining was visualized under a microscope.

### Representative Promoter Expression Results

### Zm-SARK PRO (PCO646468)

Expression was detected with the ZM-SARK PRO construct, in the bombardment of germinating seedlings, but not in leaf, or embryo bombardment or in BMS transformations.

### Zm-CCA PRO (PCO651594)

Expression was detected with the ZM-CCA PRO construct in every tissue type that was tested.

### ZM-LHY PRO:ADH1 INTRON (PCO639678)

Expression was detected with the ZM-LHY PRO construct, in the bombardment of embryos, but not in leaf or seedling bombardment or in BMS transformations.

### ZM-LHY PRO (ALT1) (PCO639678)

Expression was detected with the ZM-LHY PRO(ALT1) construct, in all bombardment experiments, but not in BMS transformations.

### ZM-NIGHT2 PRO (PCO643174)

Expression was detected with the ZM-NIGHT2 PRO construct, in the bombardment of embryos, and leaf, but not in embryo bombardment or in BMS transformations.

### ZM-NIGHT1 PRO (PCO503721)

No detectable expression was found with the ZM-NIGHT1 PRO construct in the tissue tested. It may be possible that the expression pattern, being diurnal, may not have been captured in the tested conditions.

### ZM-LICH2 PRO (PCO642613)

Expression was detected with the ZM-LICH₂ PRO construct in every tissue was tested.

### Example 4

### Transformation and Regeneration of Transgenic Plants

Immature maize embryos from greenhouse donor plants are bombarded with a plasmid containing the transformation sequence operably linked to the drought-inducible promoter RAB17 promoter (Vilardell, et al., (1990) Plant Mol Biol 14:423-432) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue:

The ears are husked and surface sterilized in 30% Clorox® bleach plus 0.5% Micro detergent for 20 minutes and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA:

A plasmid vector comprising the transformation sequence operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaC1₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment:

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment:

Following bombardment, the embryos are kept on 560Y medium for 2 days, then transferred to 560R selection medium containing 3 mg/liter Bialaphos and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media:

Bombardment medium (560Y) comprises 4.0 g/l N6 basal salts (SIGMA C-1416), 1.0 ml/I Eriksson's Vitamin Mix (1000× SIGMA-1511), 0.5 mg/l thiamine HCl, 120.0 g/l sucrose, 1.0 mg/l 2,4-D and 2.88 g/l L-proline (brought to volume with D-I H₂O following adjustment to pH 5.8 with KOH); 2.0 g/l Gelrite® (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/I Eriksson's Vitamin Mix (1000× SIGMA-1511), 0.5 mg/l thiamine HCl, 30.0 g/l sucrose and 2.0 mg/l 2,4-D (brought to volume with D-I H₂O following adjustment to pH 5.8 with KOH); 3.0 g/l Gelrite® (added after bringing to volume with D-I H₂O) and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/I MS vitamins stock solution (0.100 g nicotinic acid, 0.02 g/l thiamine HCL, 0.10 g/l pyridoxine HCL and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite® (added after bringing to volume with D-I H₂O) and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/I MS vitamins stock solution (0.100 g/l nicotinic acid, 0.02 g/l thiamine HCL, 0.10 g/l pyridoxine HCL and 0.40 g/l glycine brought to volume with polished D-I H₂O), 0.1 g/l myo-inositol and 40.0 g/l sucrose (brought to volume with polished D-I H₂O after adjusting pH to 5.6) and 6 g/l Bacto™-agar (added after bringing to volume with polished D-I H₂O), sterilized and cooled to 60° C.

### Example 5

### Agrobacterium-Mediated Transformation

For Agrobacterium-mediated transformation of maize with an antisense sequence of the transformation sequence of the present disclosure, preferably the method of Zhao is employed (U.S. Pat. No. 5,981,840 and PCT Publication Number WO 1998/32326, the contents of which are hereby incorporated by reference). Briefly, immature embryos are isolated from maize and the embryos contacted with a suspension of Agrobacterium, where the bacteria are capable of transferring the transformation sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step) and preferably calli grown on selective medium are cultured on solid medium to regenerate the plants. Plants are monitored and scored for a modulation in meristem development. For instance, alterations of size and appearance of the shoot and floral meristems and/or increased yields of leaves, flowers and/or fruits.

### Example 6

### Over Expression of Maize Diurnal Genes Affect Plant Size and Growth

The function of the diurnal gene is tested by using transgenic plants expressing the transgene. Transgene expression is confirmed by using transgene-specific primer RT-PCR.

Vegetative Growth and Biomass Accumulation:

Compared to the non transgenic sibs, the transgenic plants (in T1 generation) would be expected to show an increase in plant height. The stem of the transgenic plants is measured by comparing stem diameter values with those of non-transformed controls. The increase of the plant height and the stem thickness would result in a larger plant stature and biomass for the transgenic plants.

Diurnal genes are found to impact plant growth mainly through accelerating the growth rate but not extending the growth period. The enhanced growth, i.e., increased plant size and biomass accumulation, appears to be largely due to an accelerated growth rate and not due to an extended period of growth because the transgenic plants were not delayed in flowering based on the silking and anthesis dates. Therefore, overexpressing of the diurnal gene could accelerate the growth rate of the plant. Accelerated growth rate appears to be associated with an increased diurnal rate.

The enhanced vegetative growth, biomass accumulation in transgenics and accelerated growth rate would be further tested with extensive field experiments in both hybrid and inbred backgrounds at advanced generation (T3). Transgenic plants would be expected to show one or more of the following: increased plant height, stem diameter increases, stalk dry mass increase, increased leaf area, total plant dry mass increases.

Reproductive Growth and Grain Yield:

Overexpression of the diurnal genes would be associated with enhancing the reproductive tissue growth. T1 Transgenic plants would be expected to show one or more of the following: increased ear length, increased total kernel weight per ear, increased kernel numbers per ear and kernel size. The positive change in kernel and ear characteristics is associated with grain yield increase.

The enhanced reproductive growth and grain yield of transgenics is confirmed in extensive field experiments at the advanced generation (T3). The enhancement is observed in both inbred and hybrid backgrounds. As compared to the non-transgenic sibs as controls, the transgenic plants would be expected to show a significantly increase in one or more of the following: primary ear dry mass, secondary ear dry mass, tassel dry mass and husk dry mass.

Transgenic plants are also scored for stress tolerance parameters, including: reduced ASI, reduced barrenness and reduced number of aborted kernels. The reduction may be more when the plants are grown at a high plant density stressed condition. A reduced measurement of these parameters is often related to tolerance to biotic stress.

### Example 7

### Variants of Diurnal Sequences

A. Variant Nucleotide Sequences of Diurnal Sequences that do not Alter the Encoded Amino Acid Sequence

The diurnal nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

B. Variant Amino Acid Sequences of Diurnal Polypeptides

Variant amino acid sequences of the diurnal polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using a protein alignment, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of Diurnal Polypeptides

In this example, artificial protein sequences are created having 80%, 85%, 90% and 95% identity relative to the reference protein sequence. This latter effort requires identifying conserved and variable regions from the alignment and then the judicious application of an amino acid substitutions table. These parts will be discussed in more detail below.

Largely, the determination of which amino acid sequences are altered is made based on the conserved regions among each diurnal protein or among the other polypeptides. Based on the sequence alignment, the various regions of the polypeptide that can likely be altered are represented in lower case letters, while the conserved regions are represented by capital letters. It is recognized that conservative substitutions can be made in the conserved regions below without altering function. In addition, one of skill will understand that functional variants of the sequence of the disclosure can have minor non-conserved amino acid alterations in the conserved domain.

Artificial protein sequences are then created that are different from the original in the intervals of 80-85%, 85-90%, 90-95% and 95-100% identity. Midpoints of these intervals are targeted, with liberal latitude of plus or minus 1%, for example. The amino acids substitutions will be effected by a custom Perl script. The substitution table is provided below in Table 3.

TABLE 3
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the polypeptides are generating having about 80%, 85%, 90% and 95% amino acid identity to the starting unaltered ORF nucleotide sequence of SEQ ID NOS: 1, 3, 5 and 40-71.

### Example 8

### Alteration of Traits in Plants with the Use of Regulatory Elements and Polypeptides Disclosed Herein

The various regulatory elements including diurnal promoters and diurnal polypeptides disclosed herein are useful for a variety of trait development for crop plants. These include engineering freezing or frost tolerance, chilling or cold tolerance, drought or heat tolerance, salt stress tolerance, reduced photorespiration, stomatal aperture regulation, photosynthetic efficiency for yield increase, carbohydrate metabolism and transport, enhanced nitrogen utilization, selective metabolite biosynthesis, improved nutrient assimilation, source/sink modulation, disease resistance, insect resistance and pest resistance. One or more regulatory elements disclosed herein are combined with other regulatory elements including various stress inducible or tissue specific motifs to optimize transgene expression.

All publications and patent applications in this specification are indicative of the level of ordinary skill in the art to which this disclosure pertains. All publications and patent applications are herein incorporated by reference to the same extent as if each individual publication or patent application was specifically and individually indicated by reference.

The disclosure has been described with reference to various specific and preferred embodiments and techniques. However, it should be understood that many variations and modifications may be made while remaining within the spirit and scope of the disclosure.

What is claimed is:

1. An isolated polynucleotide selected from the group consisting of:
a. a polynucleotide having at least 90% sequence identity, as determined by the GAP algorithm under default parameters, to the full length sequence of a polynucleotide selected from the group consisting of SEQ ID NOS: 1, 2, 3, 4, 5, 6, 7, 8, 20, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470; wherein the polynucleotide encodes a polypeptide that functions as a modifier of diurnal activity;
b. a polynucleotide selected from the group consisting of SEQ ID NOS: 1, 2, 3, 4, 5, 6, 7, 8, 20, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470;
c. a polynucleotide which is fully complementary to the polynucleotide of (a) or (b);
d. a polypeptide encoded by the polynucleotide of (a) or (b); and
e. a polypeptide having at least 90% sequence identity, as determined by the GAP algorithm under default parameters, to the full length sequence of a polypeptide selected from the group consisting of SEQ ID NOS; 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 467, 469 and 471.

2. A recombinant expression cassette, comprising the polynucleotide of claim 1, wherein the polynucleotide is operably linked, in sense or anti-sense orientation, to a promoter.

3. A host cell comprising the expression cassette of claim 2.

4. A transgenic plant comprising the recombinant expression cassette of claim 2.

5. The transgenic plant of claim 4, wherein said plant is a monocot.

6. The transgenic plant of claim 4, wherein said plant is a dicot.

7. The transgenic plant of claim 4, wherein said plant is selected from the group consisting of: maize, soybean, sunflower, sorghum, canola, wheat, alfalfa, cotton, rice, barley, millet, peanut, sugar cane and cocoa.

8. A transgenic seed from the transgenic plant of claim 4.

9. A method of modulating diurnal rhythm in plants, comprising:
a. introducing into a plant cell a recombinant expression cassette comprising the polynucleotide of claim 1 operably linked to a promoter; and
b. culturing the plant under plant cell growing conditions; wherein the diurnal in said plant cell is modulated.

10. The method of claim 9, wherein the plant cell is from a plant selected from the group consisting of: maize, soybean, sunflower, sorghum, canola, wheat, alfalfa, cotton, rice, barley, millet, peanut, sugar cane and cocoa.

11. A method of modulating the whole plant or diurnal rhythm in a plant, comprising:
a. introducing into a plant cell a recombinant expression cassette comprising the polynucleotide of claim 1 operably linked to a promoter;
b. culturing the plant cell under plant cell growing conditions; and
c. regenerating a plant form said plant cell; wherein the diurnal rhythm in said plant is modulated.

12. The method of claim 11, wherein the plant is selected from the group consisting of: maize, soybean, sorghum, canola, wheat, alfalfa, cotton, rice, barley, millet, peanut and cocoa.

13. A product derived from the method of processing of transgenic plant tissues expressing an isolated polynucleotide encoding a diurnally functioning gene, the method comprising:
a. transforming a plant cell with a recombinant expression cassette comprising a polynucleotide having at least 90% sequence identity to the full length sequence of a polynucleotide selected from the group consisting of SEQ ID NO: 1, 2, 3, 4, 5, 6, 7, 8, 20, 40, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468 and 470; operably linked to a promoter; and
b. culturing the transformed plant cell under plant cell growing conditions; wherein the growth in said transformed plant cell is modulated;
c. growing the plant cell under plant-forming conditions to express the polynucleotide in the plant tissue; and
d. processing the plant tissue to obtain a product.

14. The transgenic plant of claim 13, wherein the plant is a monocot.

15. The transgenic plant of claim 13, wherein the plant is selected from the group consisting of: maize, soybean, sunflower, sorghum, canola, wheat, alfalfa, cotton, rice, barley, sugar cane and millet.

16. The transgenic plant of claim 4, where overexpression of the polynucleotide leads to which has improved plant growth as compared to non-transformed plants.

17. The transgenic plant of claim 4, where the plant exhibits improved source-sink relationships as compared to non-transformed plants.

18. The transgenic plant of claim 4, where the plant has improved yield as compared to non-transformed plants.

19. A regulatory polynucleotide molecule comprising a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.

20. A chimeric polynucleotide molecule comprising the nucleic acid fragment of claim 19.

21. The chimeric molecule of claim 20 comprises the diurnal regulatory element and a tissue specific expression element.

22. The chimeric molecule of claim 21, wherein the tissue specific expression element is selected from the group consisting of root specific, bundle sheath cell specific, leaf specific and embryo specific.

23. The regulatory polynucleotide molecule of claim 19, wherein said regulatory polynucleotide molecule is a promoter.

24. A construct comprising the regulatory molecule of claim 19 operably linked to a heterologous polynucleotide molecule, wherein the heterologous molecule confers a trait of interest.

25. The construct of claim 24, wherein the trait of interest is selected from the group consisting of drought tolerance, freezing tolerance, chilling or cold tolerance, disease resistance and insect resistance.

26. The construct of claim 24, wherein the heterologous molecule functions in source-sink metabolism.

27. A transgenic plant transformed with the regulatory molecule of claim 19.

28. The transgenic plant of claim 27 is monocotyledonous.

29. The transgenic plant of claim 27 is selected from the group consisting of maize, soybean, canola, cotton, sunflower, alfalfa, sugar beet, wheat, rye, rice, sugarcane, oat, barley, turf grass, sorghum, millet, tomato, pigeon pea, vegetable, fruit tree and forage grass.

30. A method of increasing yield of a plant, the method comprising expressing a heterologous polynucleotide of interest under the control of the regulatory molecule of claim 19.

31. The method of claim 30, wherein the heterologous polynucleotide is a diurnally regulated plant gene.

32. A method of increasing abiotic stress tolerance in a plant, the method comprising expressing one or more polynucleotides that confer abiotic stress tolerance in plants under the control of the regulatory molecule of claim 19.

33. The method of claim 32, wherein the abiotic stress tolerance is selected from the group consisting of drought tolerance, freezing tolerance and chilling or cold tolerance.

34. The method of claim 33, wherein the polynucleotide that confers drought tolerance is expressed under the control of a regulatory element whose peak expression is around mid-day or late afternoon.

35. The method of claim 33, wherein the polynucleotide that confers freezing or cold tolerance is expressed under the control of a regulatory element whose peak expression is around dawn or mid-morning.

36. A method of reducing yield drag of transgenic gene expression, the method comprising expressing a transgene operably linked to a regulatory polynucleotide molecule comprising a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.

37. A method of screening for gene candidates involved in abiotic stress tolerance, the method comprising (a) identifying one or more gene candidates that exhibit yield drag under constitutive or tissue specific expression and (b) expressing the gene candidates under the control of the a regulatory molecule that directs diurnal expression pattern.

38. The method of claim 37, wherein the regulatory molecule comprises a sequence selected from the group consisting of: (a) SEQ ID NOS: 31-183; (b) a nucleic acid fragment that comprises at least 50-100 contiguous nucleotides of one of SEQ ID NOS: 31-183 and wherein the fragment comprises one or more of the diurnal regulatory elements listed in Table 2 and (c) a nucleic acid sequence comprising at least 90% identity to about 500-1000 contiguous nucleotides of one of SEQ ID NOS: 31-183 as determined by the GAP algorithm under default parameters.
